SEQUENCE LISTING (32 sequences)

SEQ ID NO: 1 - protein, 218 aa, synthetic construct
DIQMTQSPSSLSASVGDRVTITCRASASVDSYGNSFIHWYQQKPGKAPKLLIYRASDLESGVPSRFSGSGSGTDFTLTISSLQPEDFATYYCQQNYADPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 231 aa, synthetic construct
EVQLVQSGAEVKKPGASVKVSCKASGYSITSDFAWNWVRQAPGQGLEWIGYISYSGTTSYNPSLKSRVTITRDTSTSTAYLELSSLRSEDTAVYYCARENYYGRSHVGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 3 - protein, 218 aa, synthetic construct; identical to SEQ ID NO: 1

SEQ ID NO: 4 - protein, 231 aa, synthetic construct
EVQLVESGPALVKPTQTLTLTCTFSGYSITSDFAWNWIRQPPGKALEWLAYISYSGTTSYNPSLKSRLTISKDTSKNQVVLTMTNMDPVDTATYYCARENYYGRSHVGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 5 - protein, 218 aa, synthetic construct; identical to SEQ ID NO: 1

SEQ ID NO: 6 - protein, 231 aa, synthetic construct
EVQLVESGGGLVQPGGSLRLSCAASGYSITSDFAWNWVRQAPGKGLEWVGYISYSGTTSYNPSLKSRFTISRDNSKNTLYLQMNSLRAEDTAVYYCARENYYGRSHVGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 7 - protein, 218 aa, synthetic construct; identical to SEQ ID NO: 1

SEQ ID NO: 8 - protein, 231 aa, synthetic construct
EVQLVESGPGLVKPSETLSLTCTVSGYSITSDFAWNWIRQPPGKGLEWIGYISYSGTTSYNPSLKSRVTISVDTSKNQFSLKLSSVTAADTAVYYCARENYYGRSHVGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 9 - protein, 218 aa, synthetic construct
DIVMTQTPLSLPVTPGQPASISCRASASVDSYGNSFIHWYLQKPGQSPQLLIYRASDLESGVPDRFSGSGSGTDFTLKISRVEAEDVGVYYCQQNYADPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 231 aa, synthetic construct; identical to SEQ ID NO: 2

SEQ ID NO: 11 - protein, 218 aa, synthetic construct; identical to SEQ ID NO: 9

SEQ ID NO: 12 - protein, 231 aa, synthetic construct; identical to SEQ ID NO: 4

SEQ ID NO: 13 - protein, 218 aa, synthetic construct; identical to SEQ ID NO: 9

SEQ ID NO: 14 - protein, 231 aa, synthetic construct; identical to SEQ ID NO: 6

SEQ ID NO: 15 - protein, 218 aa, synthetic construct; identical to SEQ ID NO: 9

SEQ ID NO: 16 - protein, 231 aa, synthetic construct; identical to SEQ ID NO: 8

SEQ ID NO: 17 - protein, 218 aa, synthetic construct
EIVLTQSPATLSLSPGERATLSCRASASVDSYGNSFIHWYQQKPGQAPRLLIYRASDLESGIPARFSGSGSGTDFTLTISSLEPEDFAVYYCQQNYADPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 231 aa, synthetic construct; identical to SEQ ID NO: 2

SEQ ID NO: 19 - protein, 218 aa, synthetic construct; identical to SEQ ID NO: 17

SEQ ID NO: 20 - protein, 231 aa, synthetic construct; identical to SEQ ID NO: 4

SEQ ID NO: 21 - protein, 218 aa, synthetic construct; identical to SEQ ID NO: 17

SEQ ID NO: 22 - protein, 231 aa, synthetic construct; identical to SEQ ID NO: 6

SEQ ID NO: 23 - protein, 218 aa, synthetic construct; identical to SEQ ID NO: 17

SEQ ID NO: 24 - protein, 231 aa, synthetic construct; identical to SEQ ID NO: 8

SEQ ID NO: 25 - protein, 218 aa, synthetic construct
DIVMTQSPDSLAVSLGERATINCRASASVDSYGNSFIHWYQQKPGQPPKLLIYRASDLESGVPDRFSGSGSGTDFTLTISSLQAEDVAVYYCQQNYADPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 231 aa, synthetic construct; identical to SEQ ID NO: 2

SEQ ID NO: 27 - protein, 218 aa, synthetic construct; identical to SEQ ID NO: 25

SEQ ID NO: 28 - protein, 231 aa, synthetic construct; identical to SEQ ID NO: 4

SEQ ID NO: 29 - protein, 218 aa, synthetic construct; identical to SEQ ID NO: 25

SEQ ID NO: 30 - protein, 231 aa, synthetic construct; identical to SEQ ID NO: 6

SEQ ID NO: 31 - protein, 218 aa, synthetic construct; identical to SEQ ID NO: 25

SEQ ID NO: 32 - protein, 231 aa, synthetic construct; identical to SEQ ID NO: 8

(19) United States
(12) Patent Application Publication
(10) Pub. No.: US 2024/0003900 A1
(43) Pub. Date: Jan. 4, 2024

(54) METHODS FOR PRODUCING ANTIBODIES

(71) Applicant: Genentech, Inc., South San Francisco, CA (US)

(72) Inventors: Wei-Ching LIANG, South San Francisco, CA (US); WeiYu LIN, South San Francisco, CA (US); Yan WU, South San Francisco, CA (US)

(21) Appl. No.: 18/174,889

(22) Filed: Feb. 27, 2023

Related U.S. Application Data
(63) Continuation of application No. PCT/US2021/048447, filed on Aug. 31, 2021.
(60) Provisional application No. 63/072,794, filed on Aug. 31, 2020.

(51) Int. Cl. G01N 33/68; C07K 16/28

(52) U.S. Cl. CPC G01N 33/6854; C07K 16/2803; C07K 2317/92; C07K 2317/565; G01N 33/54326

(57) ABSTRACT

Provided herein are methods for the production and identification of antibodies that bind to a desired region of a target protein.

### SEQUENCE LISTING

The instant application contains a Sequence Listing which has been submitted electronically in XML format and is hereby incorporated by reference in its entirety. Said XML copy, created on Feb. 12, 2023, is named 50474-237003_Sequence_Listing_2_12_23.xml and is 35,503 bytes in size.

### FIELD OF THE INVENTION

Provided herein are methods for the production and identification of antibodies that bind to a desired region of a target protein.

### BACKGROUND

Numerous antibody therapeutics are being advanced into pre-clinical development and clinical trials. High-quality anti-idiotypic reagent antibodies (anti-IDs), particularly complementarity-determining region (CDR)-specific anti-IDs, are critical for bioanalytical assays for therapeutic antibody drug development. Common approaches used to generate these types of reagent antibodies include in vivo hybridoma technology, in vitro phage-displayed immunized libraries, and synthetic antibody libraries. These technologies require extended timelines, and often yield panels of antibodies that are few in number, lack epitope-binding diversity, or have weak affinity. Thus, there is a need in the art for methods of rapidly producing large panels of anti-IDs having well-defined epitope-binding specificity.

### SUMMARY OF THE INVENTION

In one aspect, the disclosure features a method of identifying one or more antibodies that bind to a desired region of a target protein, the method comprising (a) providing a sample from an animal that has been immunized with the target protein or a fragment thereof comprising the desired region, wherein the sample contains IgG⁺ B cells; (b) enriching the sample for IgG⁺ B cells by separating the IgG⁺ B cells from one or more undesired cell types in the sample, wherein the separating comprises: (i) contacting the sample with one or more antibodies or antibody fragments that bind to the one or more undesired cell types, wherein the one or more antibodies or antibody fragments comprise a tag; and (ii) contacting the sample with a surface having affinity for the tag, wherein the one or more undesired cell types bound to the one or more antibodies or antibody fragments are retained on the surface, thereby separating the IgG⁺ B cells from the one or more undesired cell types and enriching the sample for IgG⁺ B cells; (c) culturing the separated IgG⁺ B cells of step (b) individually; and (d) identifying one or more IgG⁺ B cells that produce antibodies that bind to the desired region of the target protein, the identifying comprising assessing the affinity of supernatants of individually cultured IgG⁺ B cells of step (c) for both: (i) the target protein or a fragment thereof comprising the desired region; and (ii) a control protein comprising one or more undesired binding sites of the target protein or a non-target protein; wherein supernatants that have affinity for the target protein or fragment thereof and do not have affinity for the control protein identify IgG⁺ B cells producing antibodies that bind to the desired region of the target protein.

In some aspects, the animal is a rabbit or a rat. In some aspects, the animal is a rabbit.

In some aspects, the sample is a blood sample or a serum sample. In some aspects, the blood sample is a peripheral blood mononuclear cell (PBMC) sample.

In some aspects, the animal has been immunized for about 8 weeks.

In some aspects, the sample has been processed to remove macrophages and monocytes.

In some aspects, the undesired cell types are one or more of IgM B cells, myeloid cells, and T cells. In some aspects, the undesired cell types are IgM B cells, myeloid cells, and T cells. In some aspects, the one or more antibodies or antibody fragments that bind to IgM B cells are one or more anti-IgM antibodies or antibody fragments thereof that bind IgM. In some aspects, the one or more antibodies or antibody fragments that bind to myeloid cells are one or more anti-CD11 b antibodies or antibody fragments thereof that bind CD11 b. In some aspects, the one or more antibodies or antibody fragments that bind to T cells are anti-T-lymphocyte antibodies or antibody fragments thereof that bind T-lymphocytes.

In some aspects, the one or more antibodies or antibody fragments that bind to the one or more undesired cell types comprise a biotin tag and the surface comprises streptavidin.

In some aspects, the surface is a bead. In some aspects, the bead is a magnetic bead.

In some aspects, step (b) further comprises (iii) contacting the enriched sample with an antibody or antibody fragment that comprises a first marker and binds to IgG⁺ B cells and an agent that identifies viable cells.

In some aspects, the antibody or antibody fragment that binds to IgG⁺ B cells is an anti-IgG antibody.

In some aspects, the agent that identifies viable cells is propidium iodide.

In some aspects, step (b)(iii) further comprises contacting the sample with the target protein or a fragment thereof comprising the desired region, wherein the target protein or fragment thereof comprises a second marker.

In some aspects, step (b)(iii) further comprises contacting the sample with a control protein comprising one or more undesired binding sites of the target protein, wherein the control protein comprises a third marker. In some aspects, the first marker, second marker, and third marker are fluorescent markers.

In some aspects, step (b) further comprises (iv) isolating cells that are identified as viable by the agent that identifies viable cells and that comprise the first and second markers, but not the third marker. In some aspects, the isolating is by multi-parameter fluorescence activated cell sorting (FACS).

In some aspects, in step (d), an ELISA is performed for assessing the affinity of supernatants for both (i) the target protein or fragment thereof and (ii) the control protein.

In some aspects, the method further comprises (e) cloning the VH and VL regions of one or more IgG⁺ B cells that have been identified as producing antibodies that bind to the desired region of the target protein.

In some aspects, the target protein is an antibody or an antibody fragment. In some aspects, the desired region of the antibody or antibody fragment is a complementarity determining region (CDR). In some aspects, the animal has been immunized with a fragment of the antibody comprising the desired region. In some aspects, the fragment of the antibody comprising the desired region is an antigen-binding fragment (Fab).

In some aspects, the one or more undesired binding sites of the target protein are one or more framework regions of the antibody or antibody fragment. In some aspects, the control protein comprising one or more undesired binding sites of the target protein is a Fab fragment comprising: (i) a light chain (LC) comprising a framework region having at least 85% identity to the LC framework region of the target protein and a set of irrelevant LC CDRs; and (ii) a heavy chain (HC) comprising a framework region having at least 85% identity to the HC framework region of the target protein and a set of irrelevant HC CDRs. In some aspects, the irrelevant LC and HC CDRs are the CDRs of an anti-gD monoclonal antibody (mAb). In some aspects, the anti-gD mAb is 5B6.

In some aspects, the target protein is not an antibody or an antibody fragment. In some aspects, the desired region of the target protein is a domain of the target protein.

In some aspects, step (d) comprises assessing the affinity of supernatants of individually cultured IgG⁺ B cells of step (c) fora fragment of the target protein comprising the desired region. In some aspects, the fragment of the target protein comprising the desired region is linked to an irrelevant protein.

In some aspects, the control protein of step (d) is (i) a version of the target protein that is devoid of the desired region; (ii) a protein that is related to the target protein and does not comprise the desired region; or (iii) an irrelevant control protein.

In some aspects, a plurality of antibodies that bind to a desired region of a target protein is produced. In some aspects, at least 100 antibodies are produced. In some aspects, at least 500 antibodies are produced. In some aspects, at least 1,000 antibodies are produced. In some aspects, at least 10,000 antibodies are produced. In some aspects, at least 20,000 antibodies are produced. In some aspects, about 30,000 antibodies are produced.

In some aspects, at least 50% of the antibodies produced are unique.

In some aspects, the plurality of antibodies binds the desired region of the target protein with a KID of about 200 nM or lower.

In some aspects, the plurality of antibodies bind the desired region of the target protein with a KID of about 50 nM or lower. In some aspects, the plurality of antibodies bind the desired region of the target protein with a K_{D} of about 10 nM or lower. In some aspects, the plurality of antibodies bind the desired region of the target protein with a KID of about 1 nM or lower. In some aspects, the plurality of antibodies bind the desired region of the target protein with a K_{D} of about 0.1 nM or lower. In some aspects, the plurality of antibodies bind the desired region of the target protein with a KID of about 0.01 nM or lower.

In some aspects, the target protein is an antibody or an antibody fragment and the plurality of antibodies comprises at least one antigen-blocking antibody.

In some aspects, the target protein is an antibody or an antibody fragment and the plurality of antibodies comprises at least one antigen non-blocking antibody. In some aspects, the antigen non-blocking antibody binds to an antigen-antibody complex.

In some aspects, the IgG⁺ B cells of step (c) have increased viability relative to IgG⁺ B cells that have been isolated using a method that does not comprise a step of enriching the sample for IgG⁺ B cells according to the methods provided herein.

In some aspects, steps (a)-(e) are performed within twelve weeks.

### BRIEF DESCRIPTION OF THE DRAWINGS

FIG. 1A is a flow chart showing a timeline for the generation of CDR-specific anti-idiotypic antibodies (anti-IDs) from rabbits. Rabbits were immunized with a therapeutic antibody (Ab1) antigen-binding fragment (Fab) for 8 weeks; flow cytometry isolation and culturing of B cells that were IgG⁺, bound the Ab1 Fab (Ab1 Fab⁺), and did not bind a control Fab (Ab1^{ctrl} Fab⁻) from rabbit peripheral blood were performed in 1 week; primary ELISA screening, molecular cloning and rabbit antibody (rAb) expression were completed in 2 weeks; and Ab1 CDRs-specific anti-idiotypic antibodies (Ab2) were identified for characterization in 1 week.

FIG. 1B is a set of scatter plots showing enrichment of IgG⁺ B cells from immunized rabbit peripheral blood for Ab1 Fab⁺/Ab1^{ctrl} Fab⁻/IgG⁺ B cells using flow cytometry. Left panel: IgG⁺ B cells from blood. Center panel: IgG⁺ B cells following an IgG⁺ B cell enrichment step. Right panel: B cells that are IgG⁺, Ab1 Fab⁺, and Ab1^{ctrl} Fab⁻.

FIG. 1C is a graph showing the serum titer of three rabbits against the Ab1 Fab, as measured using ELISA. A pre-immunized serum sample is provided as a negative control.

FIG. 1D is a plot showing the results of a high-throughput primary ELISA screen for binding of cultured rabbit B cell supernatants to the Ab1 Fab and the Ab1^{ctrl} Fab. Optical density (OD) thresholds of OD>0.25 and OD<0.1 were used as a threshold to identify binding and non-binding supernatants, respectively.

FIG. 1E is a plot showing the results of a screen for binding of purified recombinant IgGs of 24 unique Ab2 clones to the Ab1 Fab, the Ab1^{ctrl} Fab, and another human control Fab (Huctrl Fab) derived from native IgGs in normal human plasma.

FIG. 2A is a sequence alignment diagram showing the sequence of the Ab1 light chain (LC) framework region (hIGKV1-16) with the Ab1 light chain complementarity-determining regions (CDRs) and the sequences of all four human IgG kappa LC consensus frameworks with anti-gD LC-CDRs. Dots indicate identical amino acid residues; letters indicate differing amino acid residues. Positions are numbered according to the Kabat system. CDRs are indicated according to the Kabat and Chothia definitions in IMGT®.

FIG. 2B is a sequence alignment diagram showing the sequence of the Ab1 heavy chain (HC) framework region (hIGHV3-23) and the Ab1 heavy chain CDRs and the sequences of all four human IgG HC consensus frameworks with anti-gD LC-CDRs. Dots indicate identical amino acid residues; letters indicate differing amino acid residues. Positions are numbered according to the Kabat system. CDRs are indicated according to the Kabat and Chothia definitions in IMGT®.

FIG. 3A is a schematic diagram showing an experimental setup for determining whether an Ab2 has an antigen (Ag) blocking or non-blocking epitope type. Ab2 is captured by protein A and is contacted with Ab1 Fab, followed by the antigen. Binding is assessed using surface plasmon resonance (SPR).

FIG. 3B is a pair of sensorgrams showing binding (in response units (RU)) of the Ab2 clones 3E3 and 18C9 to the Ab1 Fab and antigen. 3E3 was determined to be an Ag non-blocking anti-ID (left panel), and 18C9 was determined to be an Ag-blocking anti-ID.

FIG. 3C is a schematic diagram showing an experimental setup for determining whether an Ab2 binds to an Ab1-antigen complex. Ab2 is captured by protein A and is contacted with the Ag and Ab1 Fab complex. Binding is assessed using SPR.

FIG. 3D is a pair of sensorgrams showing binding (in response units (RU)) of the Ab2 clones 3E3 and 21A6 to the Ag and Ab1 Fab complex. 3E3 was determined to be capable of recognizing the Ag and Ab1 complex, whereas 21A6 was not.

FIG. 4 is a set of schematic diagrams showing three types of anti-IDs. Left: an Ag-blocking anti-ID (Ab2) binds the paratope of a drug antibody (Ab1), inhibiting binding of Ab1 to the target (Ag). Ag-blocking anti-IDs are useful for detection of free Ab1. Center: an Ag non-blocking anti-ID (Ab2) binds outside the drug paratope. Right: an Ag+Ab1 complex anti-ID is specific for the drug-target complex and is used exclusively for bound drug detection.

FIG. 5A is a schematic diagram showing an experimental setup for epitope binning of anti-IDs (Ab2) in a microfluidics system. The immobilized Ab2 is first bound to the Ab1 Fab, followed by the detection of pairwise Ab2 binding.

FIG. 5B is a network plot depicting epitope clusters deduced from binning the Group 2 anti-IDs (Ab2) from Project E, described herein in Example 1. Ab2 are represented by nodes. Cords indicate competing relationships between nodes. Shaded regions indicate families of Ab2 that share an identical blocking profile when tested against the other Ab2.

FIG. 5C is a network plot depicting epitope clusters deduced from binning the Group 1 anti-IDs (Ab2) from Project E. Ab2 are represented by nodes. Cords indicate competing relationships between nodes. Shaded regions indicate families of Ab2 that share an identical blocking profile when tested against the other Ab2.

FIG. 5D is a network plot depicting epitope clusters deduced from binning the Group 3 anti-IDs (Ab2) from Project E. Ab2 are represented by nodes. Cords indicate competing relationships between nodes. Shaded regions indicate families of Ab2 that share an identical blocking profile when tested against the other Ab2.

FIG. 5E is a network plot depicting epitope clusters deduced from binning a panel of 24 anti-IDs (Ab2) from Project E. Ab2 are represented by nodes. Cords indicate competing relationships between nodes. Shaded regions indicate families of Ab2 that share an identical blocking profile when tested against the other Ab2. Epitope relationships between group 1 (3E3, 14611) and group 2 (19C4) Ab2 are shown.

FIG. 6A is a schematic diagram showing an experimental setup for a sandwich ELISA PK assay. Each Ab2 was immobilized on the ELISA plate to serve as an Ab1 capture reagent, while the other Ab2 antibodies were biotinylated and conjugated to streptavidin horseradish peroxidase (HRP) to serve as the detection reagent.

FIG. 6B is a set of plots showing the results of PK assays for five Ab2 from Project E, indicating that clones 3E3 (group 1) and 19C4 (group 2) are a suitable capture and detection antibody pair.

FIG. 7A is a schematic diagram showing an experimental setup for a bridging ELISA for anti-drug antibody (ADA) development. Streptavidin-captured biotinylated Ab1 was immobilized on an ELISA plate. Ab2 were tested for bridging the captured Ab1 and a DIG-conjugated Ab1, using a mouse anti-DIG HRP conjugate as the detection reagent.

FIG. 7B is a graph showing titration of five Ab2 clones of Project E in the bridging ELISA assay shown in FIG. 7A.

FIG. 8 is an unrooted phylogenetic tree showing the differences in the CDRs of 24 unique Ab2 clones of Project E (VH:VL CDRs concatenated strings). Scale bar represents 4% sequence difference. Individual sequence differences are labelled on each branch accordingly. Clones are labeled according to the grouping designation described in Table 3.

### DETAILED DESCRIPTION OF THE INVENTION

### I. Definitions

As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) aspects that are directed to that value or parameter per se. For example, description referring to “about X” includes description of “X.” In some embodiments, “about” may refer to ±15%, ±10%, ±5%, or ±1% as understood by a person of skill in the art.

It is understood that aspects of the invention described herein include “comprising,” “consisting,” and “consisting essentially of” aspects.

The term “antibody” herein is used in the broadest sense and encompasses various antibody structures, including but not limited to monoclonal antibodies, polyclonal antibodies, multispecific antibodies (e.g., bispecific antibodies), and antibody fragments (e.g., bis-Fabs) so long as they exhibit the desired antigen-binding activity. The terms “full-length antibody,” “intact antibody,” and “whole antibody” are used herein interchangeably to refer to an antibody having a structure substantially similar to a native antibody structure or having heavy chains that contain an Fc region as defined herein.

An “antibody fragment” refers to a molecule other than an intact antibody that comprises a portion of an intact antibody that binds the antigen to which the intact antibody binds. Examples of antibody fragments include but are not limited to bis-Fabs; Fv; Fab; Fab, Fab′-SH; F(ab′)₂; diabodies; linear antibodies; single-chain antibody molecules (e.g., scFv, ScFab); and multispecific antibodies formed from antibody fragments. Antibody fragments may be produced recombinantly.

A “Fab” fragment is an antigen-binding fragment generated by papain digestion of antibodies or produced recombinantly and consists of an entire L chain along with the variable region domain of the H chain (VH), and the first constant domain of one heavy chain (CH1). Papain digestion of antibodies produces two identical Fab fragments. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having an additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” consists of a dimer of one heavy- and one light-chain variable region domain in tight, noncovalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all Lys447 residues removed, antibody populations with no Lys447 residues removed, and antibody populations having a mixture of antibodies with and without the Lys447 residue.

A “single-domain antibody” refers to an antibody fragment comprising all or a portion of the heavy chain variable domain or all or a portion of the light chain variable domain of an antibody. In certain aspects, a single-domain antibody is a human single-domain antibody (see, e.g., U.S. Pat. No. 6,248,516 B1). Examples of single-domain antibodies include but are not limited to a VHH.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthun, in The Pharmacology of Monoclonal Antibodies, vol. 113, Rosenburg and Moore eds., (Springer-Verlag, New York, 1994), pp. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of antibodies are called α, δ, ε, γ, and μ, respectively.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain aspects, such a monoclonal antibody typically includes an antibody comprising a polypeptide sequence that binds a target, wherein the target-binding polypeptide sequence was obtained by a process that includes the selection of a single target-binding polypeptide sequence from a plurality of polypeptide sequences. The monoclonal antibodies to be used in accordance with the invention may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg et al., Intern. Rev. Immunol. 13: 65-93 (1995)). It should be understood that a selected target-binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target-binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3).

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art.

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In some aspects, the extent of binding of an antibody to an unrelated target is less than about 10% of the binding of the antibody to the target as measured, e.g., by a radioimmunoassay (RIA). In some aspects, an antibody that specifically binds to a target has a dissociation constant (K_{D}) of ≤1 μM, ≤100 nM, ≤10 nM, ≤1 nM, or ≤0.1 nM. In some aspects, an antibody specifically binds to an epitope on a protein that is conserved among the protein from different species. In other aspects, specific binding can include, but does not require exclusive binding.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter. J. Mol. Biol. 227:381, 1991; Marks et al. J. Mol. Biol. 222:581, 1991. Also available for the preparation of human monoclonal antibodies are methods described in Cole et al. Monoclonal Antibodies and Cancer Therapy, Alan R. Liss, p. 77 (1985); Boerner et al. J. Immunol., 147(1):86-95, 1991. See also van Dijk and van de Winkel. Curr. Opin. Pharmacol. 5:368-74, 2001. Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al. Proc. Natl. Acad. Sci. USA. 103:3557-3562, 2006 regarding human antibodies generated via a human B-cell hybridoma technology.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al. Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al. supra. In one aspect, for the VH, the subgroup is subgroup III as in Kabat et al. supra.

A “humanized” antibody refers to a chimeric antibody comprising amino acid residues from non-human HVRs and amino acid residues from human FRs. In certain aspects, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. In certain aspects in which all or substantially all of the FRs of a humanized antibody correspond to those of a human antibody, any of the FRs of the humanized antibody may contain one or more amino acid residues (e.g., one or more Vernier position residues of FRs) from non-human FR(s). A humanized antibody optionally may comprise at least a portion of an antibody constant region derived from a human antibody. A “humanized form” of an antibody, e.g., a non-human antibody, refers to an antibody that has undergone humanization.

The term “epitope” refers to the particular site on an antigen molecule to which an antibody binds. In some aspects, the particular site on an antigen molecule to which an antibody binds is determined by hydroxyl radical footprinting. In some aspects, the particular site on an antigen molecule to which an antibody binds is determined by crystallography.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
   where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, buccal swab, sputum, tears, perspiration, mucus, tumor lysates, and tissue culture medium, tissue extracts such as homogenized tissue, tumor tissue, cellular extracts, and combinations thereof. The sample may be an archival sample, a fresh sample, or a frozen sample. In some aspects, the sample is a blood sample, e.g., a peripheral blood mononuclear cell (PBMC) sample.

### II. Methods

In some aspects, the disclosure features a method of identifying one or more antibodies that bind to a desired region of a target protein, the method comprising (a) providing a sample from an animal that has been immunized with the target protein or a fragment thereof comprising the desired region, wherein the sample contains IgG⁺ B cells; (b) enriching the sample for IgG⁺ B cells by separating the IgG⁺ B cells from one or more undesired cell types in the sample, wherein the separating comprises (i) contacting the sample with one or more antibodies or antibody fragments that bind to the one or more undesired cell types, wherein the one or more antibodies or antibody fragments comprise a tag; and (ii) contacting the sample with a surface having affinity for the tag, wherein the one or more undesired cell types bound to the one or more antibodies or antibody fragments are retained on the surface, thereby separating the IgG⁺ B cells from the one or more undesired cell types and enriching the sample for IgG⁺ B cells; (c) culturing the separated IgG⁺ B cells of step (b) individually; and (d) identifying one or more IgG⁺ B cells that produce antibodies that bind to the desired region of the target protein, the identifying comprising assessing the affinity of supernatants of individually cultured IgG⁺ B cells of step (c) for both (i) the target protein or a fragment thereof comprising the desired region; and (ii) a control protein comprising one or more undesired binding sites of the target protein or a non-target protein; wherein supernatants that have affinity for the target protein or fragment thereof and do not have affinity for the control protein identify IgG⁺ B cells producing antibodies that bind to the desired region of the target protein.

### A. Target Proteins

i. Antibody Target Proteins

In some aspects, the target protein is an antibody or an antibody fragment, e.g., a therapeutic antibody (drug antibody) or a fragment thereof. The target antibody may be, e.g., a monoclonal antibody, a polyclonal antibody, a chimeric antibody, a human antibody, a humanized antibody, a multispecific antibody (e.g., a bispecific antibody, e.g., a T-cell-dependent bispecific antibody (TDB)), and/or an antibody derivative. Antibody fragments include any molecule other than an intact antibody that comprises a portion of an intact antibody that binds the antigen to which the intact antibody binds. Examples of antibody fragments include, but are not limited to antigen-binding fragments (Fab); Fab′; Fab′-SH; F(ab′)₂ fragments; bis-Fabs; variable domains (Fv); diabodies; linear antibodies; single-chain antibody molecules (e.g., scFv, scFab); and multispecific antibodies formed from antibody fragments.

In aspects in which the target protein is an antibody or an antibody fragment, the desired region against which antibodies are generated may be any region of the target antibody or antibody fragment. In some aspects, the desired region is a complementarity-determining region (CDR) of the target antibody or antibody fragment, i.e., the antibodies generated by the method are anti-idiotypic (anti-ID) antibodies. In some aspects, the target antibody or antibody fragment comprises two or more CDRs (e.g., comprises two, three, four, five, six, or more than six CDRs), and antibodies targeting any of the CDRs are desired, e.g., the desired region comprises all of the CDRs of the target antibody or antibody fragment. In other aspects, the target antibody or antibody fragment comprises two or more CDRs and antibodies targeting only one or a subset of the CDRs are desired, e.g., the desired region comprises selected CDRs of the target antibody or antibody fragment.

The animal may be immunized with the entire target antibody or antibody fragment or with any fragment thereof comprising a properly folded version of the desired region. In some aspects, the animal is immunized with a Fab of the target antibody or antibody fragment.

ii. Non-Antibody Target Proteins

In some aspects, the target protein is not an antibody or an antibody fragment. The target protein may be any protein or peptide, e.g., a human protein or peptide, an animal protein or peptide (e.g., a cynomolgus monkey protein or peptide), a bacterial or fungal protein or peptide, or an artificial protein or peptide.

The desired region against which antibodies are generated may be any suitable region of the target protein, e.g., a domain, structure, or motif of the target protein. Exemplary domains include, but are not limited to extracellular domains, intracellular domains, transmembrane domains, and binding domains. In some aspects, the desired region of the target protein is a domain of the target protein. The animal may be immunized with any fragment of the target protein comprising a properly folded version of the desired region (e.g., domain, structure, or motif). In some aspects, the animal is immunized with the target protein. In some aspects, the animal is immunized with a protein comprising the desired region of the target protein (e.g., domain, structure, or motif) linked to an irrelevant protein. Irrelevant proteins include proteins that do not have a domain, structure, or motif with structural or functional similarity to the desired region of the target protein. In some aspects, linking the desired region to an irrelevant protein allows for the generation of antibodies against a properly folded version of the desired region in the absence of other domains, structures or motifs of the target protein. The irrelevant protein may be used as a negative selection screen to eliminate antibodies that bind to it. In some aspects, antibodies generated by the methods described herein are species-specific, e.g., bind specifically to a target protein of a species of interest and do not bind to a related protein (e.g., a homolog of the target protein) of an undesired species. In some aspects, an antibody generated by the methods described herein binds to a human target protein, but not to a related mouse protein (e.g., a mouse homolog). In some aspects, an antibody generated by the methods described herein binds to a human target protein and a related cynomolgus (cyno) protein (e.g., a cyno homolog), but not to a related mouse protein (e.g., a mouse homolog).

### B. Samples Containing IgG⁺ B Cells

Immunization may be performed in and immunized samples may be provided from any suitable animal, e.g., a mammal, e.g., a rat, rabbit, hamster, or mouse. In some aspects, the animal is a rabbit or a rat. In some aspects, the animal is a rabbit.

In some aspects, the animal has been immunized with the target protein or a fragment thereof comprising the desired region for between about six and about fifteen weeks, e.g., has been immunized for about one week, two weeks, three weeks, four weeks, five weeks, six weeks, seven weeks, eight weeks, nine weeks, ten weeks, eleven weeks, twelve weeks, thirteen weeks, fourteen weeks, or fifteen weeks (e.g., has been immunized for about six weeks to about ten weeks). In some aspects, the animal has been immunized for about eight weeks. The immunization may comprise multiple administrations of the target protein or fragment thereof comprising the desired region.

The sample from the immunized animal containing IgG⁺ B cells may be, e.g., a blood sample or a serum sample. In some aspects, the blood sample is a peripheral blood mononuclear cell (PBMC) sample.

In some aspects, a sample (e.g., a blood sample, e.g., a PBMC sample) is provided from a human who has been vaccinated with a specific antigen, has survived a disease, or has a disease.

In some aspects, the sample from the immunized animal (e.g., blood sample, e.g., PMBC sample) has been processed to remove macrophages and/or monocytes from the sample. Exemplary methods for removing macrophages and monocytes from a sample by non-specific adhesion onto a plate are described in Seeber et al. PLoS One, 9: e86184, 2014, which is incorporated by reference herein in its entirety.

### C. Methods of Enriching Samples for IgG⁺ B Cells

In some aspects, the disclosure features a method of enriching a sample for IgG⁺ B cells by separating the IgG⁺ B cells from one or more undesired cell types a sample from an animal, wherein the separating comprises (i) contacting the sample with one or more antibodies or antibody fragments that bind to the one or more undesired cell types, wherein the one or more antibodies or antibody fragments comprise a tag; and (ii) contacting the sample with a surface having affinity for the tag, wherein the one or more undesired cell types bound to the one or more antibodies or antibody fragments are retained on the surface, thereby separating the IgG⁺ B cells from the one or more undesired cell types and enriching the sample for IgG⁺ B cells.

In some aspects, the undesired cell types are one or more undesired cell types present in a sample from an animal, e.g., a blood sample or a plasma sample. In some aspects, the undesired cell types include one, two, or all three of IgM B cells, myeloid cells, and T cells. In some aspects, the undesired cell types are IgM B cells, myeloid cells, and T cells.

In some aspects, the undesired cell types include IgM B cells, and the method includes contacting the sample with one or more antibodies or antibody fragments that bind to IgM B cells, e.g., one or more anti-IgM antibodies or antibody fragments thereof that bind IgM.

In some aspects, the undesired cell types include myeloid cells, and the method includes contacting the sample with one or more antibodies or antibody fragments that bind to myeloid cells, e.g., one or more anti-CD11 b antibodies or antibody fragments thereof that bind CD11 b.

In some aspects, the undesired cell types include T cells, and the method includes contacting the sample with one or more antibodies or antibody fragments that bind to T cells, e.g., one or more anti-T-lymphocyte antibodies or antibody fragments thereof that bind T-lymphocytes.

In some aspects, the IgG⁺ B cells are separated from at least 5% of cells of the undesired cell type in the sample (e.g., IgM B cells, myeloid cells, and/or T cells), e.g., are separated from at least 10%, 15%, 20%, 25%, 30%, 35%, 40%, 45%, 50%, 55%, 60%, 65%, 70%, 75%, 80%, 85%, 90%, 95%, 97%, 98%, or 99% or are separated from 100% of cells of the undesired cell type in the sample, e.g., are separated from 5%-25%, 25%-50%, 50%-70%, 70%-80%, 80%-90%, 90%-95%, 95%-98%, or 98%-100% of cells of the undesired cell type in the sample. In some aspects, the IgG⁺ B cells are separated from at least between 50% and 70% of cells of the undesired cell type in the sample. In some aspects, the IgG⁺ B cells are separated from at least 98% of cells of the undesired cell type in the sample.

The tag comprised by the one or more antibodies or antibody fragments that bind to the one or more undesired cell types (e.g., anti-IgM antibodies, anti-CD11 b antibodies, anti-T-lymphocyte antibodies, and/or fragments thereof) may be any suitable tag. In some aspects, the tag is a biotin tag.

The surface having affinity for the tag may be, e.g., a surface comprising a moiety having affinity for the tag. In some aspects, the tag is a biotin tag and the surface comprises streptavidin. In some aspects, the surface is a bead, e.g., a magnetic bead. The surface (e.g., bead, e.g., magnetic bead)) may be a component of a column purification system. Contacting the sample with the surface having affinity for the tag may comprise, e.g., flowing the sample over the surface (e.g., flowing the sample through a column purification system comprising the surface).

D. Methods of Selecting IgG⁺ B Cells with Desired Target Specificity

i. Methods of Selecting Viable, IgG⁺ Cells

In some aspects of the methods described herein, step (b) of the method further comprises (iii) contacting the enriched sample with an antibody or antibody fragment that comprises a first marker and binds to IgG⁺ B cells and an agent that identifies viable cells.

In some aspects, the antibody or antibody fragment that comprises a first marker and binds to IgG⁺ B cells is an anti-IgG antibody. The first marker may be, e.g., a fluorescent marker. In some aspects, the first marker is fluorescein isothiocyanate (FITC).

In some aspects, the agent that identifies viable cells is propidium iodide (PI). PI stains non-viable cells; thus, a relatively low level of PI staining (e.g., absence of PI staining or a level of PI staining that is below a reference level) may be used to identify a cell as viable. Other methods and agents that may be used to identify viable cells include, but are not limited to ethidium homodimer assays, TUNEL assays, Evans blue staining, fluorescein diacetate (FDA) hydrolysis assays, formazan dye staining, MTT assays, neutral red staining, resazurin staining, Janus Green B staining, 7-AAD staining, and trypan blue staining.

In some aspects, the method further comprises selecting cells that are identified as IgG⁺ based on detection of the first marker (e.g., detection of a fluorescent signal from the first marker that is above a reference level) and are identified as viable by the agent that identifies viable cells (e.g., identified as viable based on a level of PI staining that is below a reference level), e.g., separating such cells from the sample.

In some aspects, the first marker and the agent that identifies viable cells (e.g., PI) are fluorescent markers, and flow cytometry is used to assess the fluorescent signals from the marker and the agent for individual cells. In some aspects, the flow cytometry is fluorescence-activated cell sorting (FACS), and cells that are identified as IgG⁺ and viable are selected and separated from the sample using FACS.

ii. Methods of Selecting IgG⁺ B Cells with Desired Target Specificity

In some aspects of the methods described herein, step (b)(iii) further comprises contacting the sample with the target protein or a fragment thereof comprising the desired region, wherein the target protein or fragment thereof comprises a second marker. The second marker may be, e.g., a fluorescent marker. In some aspects, the second marker is R-phycoerythrin (RPE).

In some aspects, the method further comprises selecting cells that are identified as IgG⁺ based on detection of the first marker; are identified as viable by the agent that identifies viable cells; and are identified as binding the target protein or fragment thereof based on detection of the second marker (e.g., detection of a fluorescent signal from the second marker that is above a reference level), e.g., separating such cells from the sample.

In some aspects of the methods described herein, step (b)(iii) further comprises contacting the sample with a control protein comprising one or more undesired binding sites of the target protein, wherein the control protein comprises a third marker. The third marker may be, e.g., a fluorescent marker. In some aspects, the third marker is allophycocyanin (APC).

In some aspects, the method further comprises selecting cells that are identified as IgG⁺ based on detection of the first marker; are identified as viable by the agent that identifies viable cells; and are identified as binding the target protein or fragment thereof based on detection of the second marker; and are identified as not binding the control protein based on detection of the third marker (e.g., absence of the third marker or detection of a fluorescent signal from the third marker that is below a reference level), e.g., separating such cells from the sample.

In some aspects of the methods described herein, step (b) further comprises (iv) isolating cells that are identified as viable by the agent that identifies viable cells and that comprise the first and second markers, but not the third marker. In some aspects, the isolating is by multi-parameter fluorescence activated cell sorting (FACS).

In some aspects, the first marker, the second marker, the third marker, and the agent that identifies viable cells (e.g., PI) are fluorescent markers having distinguishable emission spectra, and flow cytometry is used to assess the fluorescent signals from the markers and the agent for individual cells. In some aspects, the flow cytometry is fluorescence-activated cell sorting (FACS), and cells that are identified as IgG⁺, viable, binding to the target protein or fragment thereof, and not binding to the control protein are selected and separated from the sample using FACS.

In some aspects, the disclosure features a method of isolating an IgG⁺ B cell having a desired target specificity, the method comprising (a) providing a sample from an animal that has been immunized with the target protein or a fragment thereof comprising the desired region, wherein the sample contains IgG⁺ B cells, (b) contacting the sample with an agent that identifies viable cells; an antibody or antibody fragment that comprises a first marker and binds to IgG⁺ B cells; the target protein or a fragment thereof comprising the desired region, wherein the target protein or fragment thereof comprises a second marker; and a control protein comprising one or more undesired binding sites of the target protein, wherein the control protein comprises a third marker; and (c) isolating cells that are identified as viable by the agent that identifies viable cells and that comprise the first and second markers, but not the third marker.

### E. Control Proteins

In some aspects, the methods described herein involve use of a negative control including a protein region, domain, structure, or motif to which binding by the antibodies is not desired, e.g., one or more undesired binding sites of the target protein or one or more undesired binding sites of a non-target protein, to identify antibodies having undesired specificity. In some aspects, the undesired binding site is present in the target protein or fragment thereof with which the animal has been immunized. In other aspects, the undesired binding site is not present in the protein or fragment thereof used for immunization.

i. Antibody Control Proteins

In aspects in which the target protein is an antibody or an antibody fragment, the one or more undesired binding sites of the target protein may be, e.g., one or more framework regions or one or more constant regions of the antibody or antibody fragment.

In some aspects, the target protein is an antibody or an antibody fragment and the one or more undesired binding sites of the target protein are one or more framework regions of the antibody or antibody fragment. In some aspects, the target antibody or antibody fragment comprises two or more framework regions (e.g., comprises two, three, four, five, six, seven, eight, or more than eight framework regions), and the undesired binding sites comprise all of the framework regions of the target antibody or antibody fragment. In other aspects, the undesired binding sites comprise one or a subset of the framework regions of the target antibody or antibody fragment.

In some aspects, the control protein comprising one or more undesired binding sites of the target protein is a Fab fragment comprising (i) a light chain (LC) comprising a framework region having at least 80% identity (e.g., at least 81%, 82%, 83%, 84%, 85%, 86%, 87%, 88%, 89%, 90%, 91%, 92%, 93%, 94%, 95%, 96%, 97%, 98%, or 99% identity or 100% identity) to the LC framework region of the target antibody or antibody fragment and a set of irrelevant LC CDRs; and (ii) a heavy chain (HC) comprising a framework region having at least 80% identity (e.g., at least 81%, 82%, 83%, 84%, 85%, 86%, 87%, 88%, 89%, 90%, 91%, 92%, 93%, 94%, 95%, 96%, 97%, 98%, or 99% identity or 100% identity) to the HC framework region of the target antibody or antibody fragment and a set of irrelevant HC CDRs.

In some aspects, the control protein comprising one or more undesired binding sites of the target protein is a Fab fragment comprising (i) a light chain (LC) comprising a framework region having at least 85% identity to the LC framework region of the target protein and a set of irrelevant LC CDRs; and (ii) a heavy chain (HC) comprising a framework region having at least 85% identity to the HC framework region of the target protein and a set of irrelevant HC CDRs.

Irrelevant LC and HC CDRs may, for example, be CDRs of any antibody that does not bind to the epitope of the target antibody or antibody fragment. Alternatively, irrelevant LC and HC CDRs may, for example, be CDRs of an antibody that binds to the epitope of the target antibody or antibody fragment, wherein the CDRs do not share substantial sequence similarity with the CDRs of the target antibody, e.g., have less than 70% identity with the CDRs of the target antibody (e.g., less than 65%, 60%, 55%, 50%, 45%, 40%, 35%, 30%, 25%, 20%, 15%, 10%, or 5% identity). In some aspects, the irrelevant LC and HC CDRs are the CDRs of an anti-gD monoclonal antibody (mAb). In some aspects, the anti-gD mAb is 5B6. In some aspects, the irrelevant LC or HC CDR is a CDR fragment provided in Table 1.

TABLE 1
Control Fab fragments

| | | |
|---|---|---|
| K1H1 | LC | SEQ ID NO: 1 |
| | HC | SEQ ID NO: 2 |
| K1H2 | LC | SEQ ID NO: 3 |
| | HC | SEQ ID NO: 4 |
| K1H3 | LC | SEQ ID NO: 5 |
| | HC | SEQ ID NO: 6 |
| K1H4 | LC | SEQ ID NO: 7 |
| | HC | SEQ ID NO: 8 |
| K2H1 | LC | SEQ ID NO: 9 |
| | HC | SEQ ID NO: 10 |
| K2H2 | LC | SEQ ID NO: 11 |
| | HC | SEQ ID NO: 12 |
| K2H3 | LC | SEQ ID NO: 13 |
| | HC | SEQ ID NO: 14 |
| K2H4 | LC | SEQ ID NO: 15 |
| | HC | SEQ ID NO: 16 |
| K3H1 | LC | SEQ ID NO: 17 |
| | HC | SEQ ID NO: 18 |
| K3H2 | LC | SEQ ID NO: 19 |
| | HC | SEQ ID NO: 20 |
| K3H3 | LC | SEQ ID NO: 21 |
| | HC | SEQ ID NO: 22 |
| K3H4 | LC | SEQ ID NO: 23 |
| | HC | SEQ ID NO: 24 |
| K4H1 | LC | SEQ ID NO: 25 |
| | HC | SEQ ID NO: 26 |
| K4H2 | LC | SEQ ID NO: 27 |
| | HC | SEQ ID NO: 28 |
| K4H3 | LC | SEQ ID NO: 29 |
| | HC | SEQ ID NO: 30 |
| K4H4 | LC | SEQ ID NO: 31 |
| | HC | SEQ ID NO: 32 |

ii. Non-Antibody Control Proteins

In aspects in which the target protein is not an antibody or an antibody fragment, the control protein may be, e.g., (i) a version of the target protein that is devoid of the desired region (e.g., a version of the target protein that is devoid of (e.g., has been modified to lack) one or more domains, structures, or motifs comprising the desired region or a version of the target protein in which the amino acids comprising the desired region have been replaced with irrelevant amino acids); (ii) a protein that is related to the target protein and does not comprise the desired region (e.g., an ortholog or homolog of the target protein); or (iii) an irrelevant control protein. Irrelevant control proteins include proteins that do not have a domain, structure, or motif with structural or functional similarity to the desired region of the target protein, e.g., proteins in the family of the target protein that do not have such a domain, structure, or motif.

### F. B Cell Culture

In some aspects of the methods described herein, the method comprises step (c), culturing the separated IgG⁺ B cells of step (b) individually. In some aspects, the cells are cultured in conditioned medium, e.g., rbTSN. In some aspects, the cells are cultured in conditioned medium with feeder cells, e.g., as described in Seeber et al., PLoS One, 9: e86184, 2014 and in WO 2013/076139, which is incorporated by reference herein in its entirety.

In some aspects, the survival rate of IgG⁺ B cells in the culturing step is at least 40%, e.g., is at least 45%, 50%, 55%, 60%, 65%, 70%, 75%, 80%, 85%, 90%, or 95% or is greater than 95%. In some aspects, the survival rate of IgG⁺ B cells is between about 50% and 80%.

G. Methods of Identifying IgG⁺ B Cells that Produce Antibodies that Bind to a Desired Region of a Target Protein

In some aspects of the methods described herein, step (d) of the method comprises performing an enzyme-linked immunosorbent assay (ELISA) for assessing the affinity of supernatants for both (i) the target protein or fragment thereof and (ii) the control protein (e.g., a control protein as described in Section IIE herein). Appropriate cutoff thresholds (e.g., optical density (OD) thresholds) for individual experiments may be determined based on, e.g., the concentration of antibodies in the supernatants, the level of background binding, and the number of clones being screened.

### H. Methods of Cloning IgG⁺ B Cells

In some aspects of the methods provided herein, the method further comprises (e) cloning the VH and VL regions of one or more IgG⁺ B cells that have been identified as producing antibodies that bind to the desired region of the target protein.

### I. Properties of Antibody Libraries

In some aspects of the methods described herein, a plurality of antibodies that bind to the desired region of the target protein is produced. In some aspects, at least 5, 10, 15, 20, 25, 30, 35, 40, 45, 50, 55, 60, 65, 70, 75, 80, 85, 90, 95, or 100 antibodies (e.g., 5-25, 25-50, 50-75, or 75-100 antibodies) are produced. In some aspects, at least 100 antibodies are produced. In some aspects, at least 150, 200, 250, 300, 350, 400, 450, or 500 antibodies (e.g., 150-250, 250-350, 350-450, or 450-500 antibodies) are produced. In some aspects, at least 500 antibodies are produced. In some aspects, at least 550, 600, 650, 700, 750, 800, 850, 900, 950, or 1000 antibodies (e.g., 550-650, 650-750, 750-850, 850-950, or 950-1000 antibodies) are produced. In some aspects, at least 1000 antibodies are produced. In some aspects, at least 1500, 2000, 2500, 3000, 3500, 4000, 4500, 5000, 5500, 6000, 6500, 7000, 7500, 8000, 8500, 9000, 9500, or 10,000 antibodies (e.g., 1500-2500, 2500-3500, 3500-4500, 4500-5500, 5500-6500, 6500-7500, 7500-8500, 8500-9500, or 9500-10,000 antibodies) are produced. In some aspects, at least 10,000 antibodies are produced. In some aspects, at least 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 antibodies (e.g., 10,500-11,500, 11,500-12,500, 12,500-13,500, 13,500-14,500, 14,500-15,500, 15,500-16,500, 16,500-17,500, 17,500-18,500, 18,500-19,500, or 19,500-20,000 antibodies) are produced. In some aspects, at least 20,000 antibodies are produced. In some aspects, at least 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, or 30,000 antibodies (e.g., 20,500-21,500, 21,500-22,500, 22,500-23,500, 23,500-24,500, 24,500-25,500, 25,500-26,500, 26,500-27,500, 27,500-28,500, 28,500-29,500, or 29,500-30,000 antibodies) are produced. In some aspects, at least 30,000 antibodies are produced.

In some aspects of the methods described herein, at least 50% of the antibodies produced (e.g., at least 55%, 60%, 65%, 70%, 75%, 80%, 85%, 90%, 95%, 98%, or 99% of the antibodies produced) are unique.

In some aspects, the plurality of antibodies (e.g., at least a subset of the plurality of antibodies, e.g., at least 5%, 10%, 15%, 20%, 25%, 30%, 35%, 40%, 45%, 50%, 55%, 60%, 65%, 70%, 75%, 80%, 85%, 90%, 95%, 98%, or 99% of the antibodies) bind the desired region of the target protein with a KID of about 200 nM or lower, e.g., about 175 nM or lower, 150 nM or lower, 125 nM or mower, 100 nM or lower, or 75 nM or lower. In some aspects, the subset of the plurality of antibodies binds the desired region of the target protein with a KID of about 50 nM or lower, e.g., about 45 nM or lower, 40 nM or lower, 35 nM or lower, 30 nM or lower, 25 nM or lower, 20 nM or lower, or 15 nM or lower. In some aspects, the subset of the plurality of antibodies binds the desired region of the target protein with a KID of about 10 nM or lower, e.g., about 9 nM or lower, 8 nM or lower, 7 nM or lower, 6 nM or lower, 5 nM or lower, 4 nM or lower, 3 nM or lower, 2 nM or lower, or 1.5 nM or lower. In some aspects, the subset of the plurality of antibodies binds the desired region of the target protein with a KID of about 1 nM or lower, e.g., about 0.9 nM or lower, 0.8 nM or lower, 0.7 nM or lower, 0.6 nM or lower, 0.5 nM or lower, 0.4 nM or lower, 0.3 nM or lower, 0.2 nM or lower, or 0.15 nM or lower. In some aspects, the subset of the plurality of antibodies binds the desired region of the target protein with a KID of about 0.1 nM or lower, e.g., about 0.09 nM or lower, 0.08 nM or lower, 0.07 nM or lower, 0.06 nM or lower, 0.05 nM or lower, 0.04 nM or lower, 0.03 nM or lower, 0.02 nM or lower, or 0.015 nM or lower. In some aspects, the subset of the plurality of antibodies binds the desired region of the target protein with a KID of about 0.01 nM or lower, e.g., about 0.009 nM or lower, 0.008 nM or lower, 0.007 nM or lower, 0.006 nM or lower, 0.005 nM or lower, 0.004 nM or lower, 0.003 nM or lower, 0.002 nM or lower, or 0.001 nM or lower.

In some aspects in which the target protein is an antibody or an antibody fragment, the plurality of antibodies comprises at least one antigen-blocking antibody (e.g., at least 2, 3, 4, 5, 10, 15, 20, 25, 50, 100, or more than 100 antigen-blocking antibodies). Antigen-blocking antibodies bind to the paratope of the target antibody or antibody fragment and interfere with antigen binding, and are thus useful for the detection of free target antibody or antibody fragment.

In some aspects in which the target protein is an antibody or an antibody fragment, the plurality of antibodies comprises at least one antigen non-blocking antibody (e.g., at least 2, 3, 4, 5, 10, 15, 20, 25, 50, 100, or more than 100 antigen non-blocking antibodies). Antigen non-blocking antibodies bind outside of the paratope of the target antibody or antibody fragment and do not block binding of the antigen, and are thus useful for the detection of free target antibody or antibody fragment and possibly for the detection of the target antibody or antibody fragment partially or fully bound by the antigen. In some aspects, the antigen-blocking antibody binds to the antigen-antibody complex. In other aspects, the antigen-blocking antibody does not bind to the antigen-antibody complex.

In some aspects of the methods provided herein, the IgG⁺ B cells of step (c) have increased viability relative to IgG⁺ B cells that have been isolated using a method that does not comprise a step of enriching the sample for IgG⁺ B cells according the methods provided herein, e.g., have viability that is increased by at least 5%, 10%, 15%, 20%, 25%, 30%, 35%, 40%, 45%, 50%, 55%, 60%, 70%, 75%, 80%, 85%, 90%, or 95% relative to IgG⁺ B cells that have been isolated using a method that does not comprise a step of enriching the sample for IgG⁺ B cells according the methods provided herein.

In some aspects of the methods provided herein, steps (a)-(e) are performed within about ten to fourteen weeks, e.g., performed within about ten weeks, eleven weeks, twelve weeks, thirteen weeks, or fourteen weeks. In some aspects, steps (a)-(e) are performed within twelve weeks.

### III. Examples

The following are examples of methods, uses, and compositions of the invention. It is understood that various other aspects may be practiced, given the general description provided above, and the examples are not intended to limit the scope of the claims.

### Example 1. Efficient Workflow to Isolate Ab1-Specific Single Rabbit IgG⁺ B Cells for In Vitro Clonal Expansion Culture

An optimized rabbit single B cell sorting-culture and cloning method for therapeutic antibody (Ab1) CDRs-specific anti-ID (Ab2) discovery was developed, which included four phases: (1) IgG⁺ B cell enrichment before sorting, (2) negative selection using a designed Ab1 framework control Fab (Ab1^{ctrl} Fab) as a “negative gate” to exclude Ab1 framework-specific B cells, (3) use of an integrated robotic system to enhance screening throughput of B cells cultured supernatants, and (4) preserved B cells in screening for follow-up rapid cloning and recombinant IgG expression. This approach allows the generation of large panels of anti-IDs routinely and with a high degree of success. Compared to previous antibody discovery approaches, the new platform consistently and effectively delivers high-affinity anti-IDs with a high degree of Ab1-CDR specificity and more diverse epitopes in a fraction of the time, providing tangible benefits to bioanalytical programs supporting recombinant therapeutic antibody (Ab1) projects.

Using this novel approach, 11 projects using unique Ab1s were successfully accomplished, with the generation of anti-IDs having high sequence diversities (>55%) and broad affinity range (low pM to high nM), regardless of the number of clones being identified in the process (Table 2). The recombinant anti-IDs were available for assay development to support pharmacokinetic and immunogenicity studies within twelve weeks from the start of rabbit immunization. Herein, details related to Project E are highlighted as an illustrative example.

TABLE 2
Framework control, molecular cloning and binding affinity summary in multiple anti-IDs project campaigns

| | Ab1 light chain | | | Ab1 heavy chain | | | Ab1^{Ctrl} | Anti-IDs characterization | | | |
|---|---|---|---|---|---|---|---|---|---|---|---|
| | | Closest | | | Closest | | Human | Ab 1 | | | |
| | Human | human | | Human | human | | consensus | CDRs | Molecular cloning | | Affinity |
| | framework | consensus | Identity | framework | consensus | Identity | framework | specific | | Uniqueness | range |
| Project | germline | framework | (%) | germline | framework | (%) | control | clones | Cloned | (%) | (K_{D}:nM) |
| A | hIGKV1-39 | K1 | 98 | hIGKV1-46 | H1 | 98 | K1H1 | 146 | 96 | 78 | 0.002-71 |
| B | hIGKV4-01 | K4 | 99 | hIGKV1-46 | H1 | 99 | K4H1 | 75 | 75 | 59 | 0.04-8 |
| C | hIGKV1-16 | K1 | 99 | hIGKV4-59 | H4 | 93 | K1H4 | 901 | 48 | 88 | 0.01-44 |
| D | hIGKV4-01 | K4 | 99 | hIGKV1-03 | H1 | 99 | K4H1 | 48 | 48 | 55 | 0.003-4 |
| E | hIGKV1-16 | K1 | 100 | hIGKV3-23 | H3 | 98 | K1H3 | 34 | 34 | 71 | 0.004-6 |
| F | hIGKV1-05 | K1 | 93 | hIGKV1-02 | H1 | 89 | K1H1 | 48 | 48 | 83 | 0.03-202 |
| G | hIGKV1-16 | K1 | 96 | hIGKV3-48 | H3 | 99 | K1H3 | 37 | 37 | 76 | 0.15-4 |
| H | hIGKV1-12 | K1 | 93 | hIGKV1-46 | H1 | 98 | K1H1 | 796 | 48 | 92 | 0.016-1 |
| I | hIGKV1-39 | K1 | 98 | hIGKV1-46 | H1 | 99 | K1H1 | 79 | 42 | 69 | 0.007-167 |
| J | hIGKV1-12 | K1 | 100 | hIGKV3-23 | H3 | 96 | K1H3 | 659 | 48 | 81 | 0.001-2 |
| K | hIGKV2-28 | K2 | 98 | hIGKV3-23 | H3 | 96 | K2H3 | 967 | 96 | 75 | 0.006-8 |

The framework germlines in 11 unique Ab1 projects and their corresponding closest human consensus framework control chosen to guide selection are listed. The identity (%) of light and heavy chain for each project were determined from sequence alignment of Ab1 framework germline with the closest human consensus framework. Part or all of the Ab1 CDRs-specific anti-IDs were molecular cloned to determine their sequence uniqueness shown in %. The unique clone here was defined by >10% amino acid difference in CDRs sequences. The binding affinities for all unique anti-IDs against Ab1 Fab at each project were measured by Biacore SPR and listed as a range from low pM to high nM.

A. Rabbit Immunization and Polyclonal Sera Titer Check

New Zealand White (NZVV) rabbits purchased from Western Oregon Rabbit Company (WORC) were immunized with the antigen-binding fragment of a target antibody (Ab1 Fab) in a local contract research organization. Three rabbits in each project were immunized with Ab1 Fab (500 μg) formulated with Complete Freund's adjuvant (CFA) in a 1:1 mixture through subcutaneous (SC) and intradermal (ID) injections along the back of the rabbit. Three additional boosts of Ab1 Fab (250 μg), formulated with Incomplete Freund's adjuvant (IFA) in a 1:1 mixture, were administered by SC injection three weeks after primary immunization. The Fab-immunogen approach was chosen not only to avoid unwanted reactivity against the constant regions of the antibody via presentation of constant HC, CH2, and CH3 as antigenic determinants, but also to prevent Fc sequences from triggering non-specific interactions during rabbit B cell sorting.

Standard ELISA protocol was used to monitor anti-Ab1 Fab polyclonal sera titer during the immunization period, as follows: first, a 96-well NUNC™ MICROWELL™ microtiter plate was coated with Ab1 Fab (1 μg/mL) in a coating buffer (0.05 M sodium carbonate, pH 9.6) overnight at 4° C. The plate was then blocked with assay buffer (1× PBS, 0.5% BSA and 0.05% polysorbate 20) before incubating with serial dilution of rabbit serum for 1 hour. Binding was detected using a goat anti-rabbit IgG conjugated to horseradish peroxidase (12-348, Sigma) with a TMB substrate (Surmodics, Inc.), and the reaction was ceased by Stop Solution (BSTP-1000-01, Surmodics, Inc.) after 5 minutes for optical density (OD) reading at 650 nm.

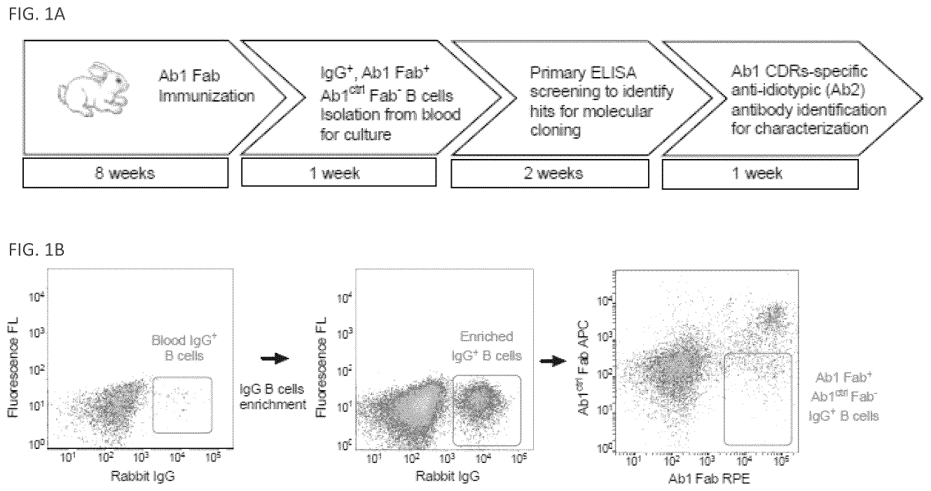

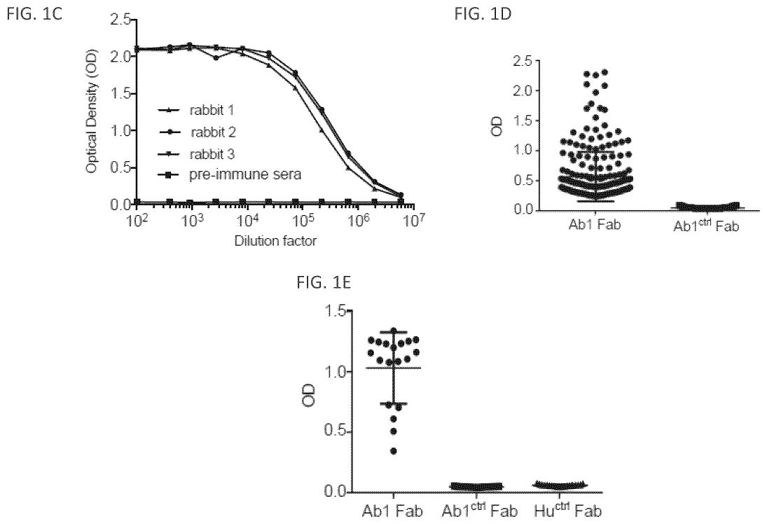

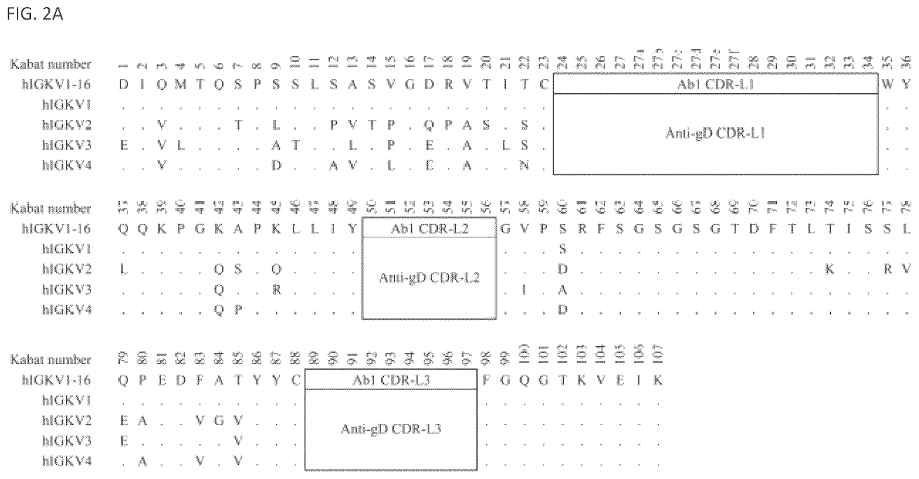

In Project E, the workflow started with eight weeks immunization with Ab1 Fab (FIG. 1A), and three rabbits demonstrated strong anti-Ab1 serum IgG titer, with positive binding at up to a 1:1,000,000 dilution factor, demonstrating robust Ab1 immunization (FIG. 1C).

B. PBMC Isolation and IgG⁺ B Cell Enrichment

This example describes a method to enrich IgG⁺ B cells before multi-parameter fluorescence activated cell sorting (FACS), thus improving the efficiency of identification of antigen-specific IgG⁺ B cells from peripheral blood mononuclear cells (PBMCs) and shortening the FACS sorting process time.

The purpose of this approach was to eliminate non-IgG⁺ B cells, including IgM B cells, myeloid cells, and T cells, from PBMCs using MACS beads-based negative-selection strategy. This negative-selection enrichment approach is more efficient than “dump channel” selection during FACS sorting to exclude non-IgG B cells and is believed to avoid potential activation-induced cell death. The method increased the IgG⁺ B cell population up to 25-fold, and also reduced the sorting time (3-30 minutes per plate versus 30-90 minutes per plate), potentially improving B cell survival rate.

Rabbit peripheral blood mononuclear cells (PBMCs) were isolated by density centrifugation of blood (1:1 dilution with PBS) collected from a rabbit ear artery using LYMPHOLYTE®-M (CL5030, CEDARLANE®). After washing with PBS, PBMCs were resuspended in culture medium RPMI with supplement and transferred to a 6-well plate to remove macrophages and monocytes through non-specific adhesion onto the plate, as described in Seeber et al., PLoS One, 9: e86184, 2014. The non-adhered cells were then collected for B cell enrichment. The sample was incubated with an antibody cocktail containing commercially available biotinylated antibodies with highly selective profiles: an anti-rabbit CD11 b antibody (MCA802GA, Bio-Rad), an anti-rabbit T-lymphocyte antibody (MCA800GA, BioRad), and an anti-rabbit IgM antibody (550938, BD Bioscience). The sample was then passed through a MACS® Column (130-042-401, Miltenyi Biotec) in the presence of streptavidin-coated magnetic beads. Only the cells that are bound to the biotinylated antibodies were attached to the beads when passed through the column with applied magnetic field: rabbit myeloid cells, T cells, and IgM B cells were thus depleted from the sample by the process. The unbound cells, including IgG⁺ B cells, were able to pass through the column, and were thus enriched by the process (FIG. 1B).

C. Ab1 CDR-Specific IgG⁺ B Cell Single-Cell Sorting and Culture

Enriched rabbit IgG⁺ B cells were stained with a FITC-labeled goat anti-rabbit IgG antibody (STAR121F, Bio-Rad) and contacted with RPE-labeled Ab1 Fab and APC-labeled Ab1^{ctrl} Fab (Example 2) in the staining buffer (PBS with 2% FBS) for 20 minutes at 4° C. Prior to fluorescence-activated cell sorting (FACS sorting), cells were washed and resuspended in staining buffer containing 5 μg/mL propidium iodide (PI; 556463, BD Biosciences) to allow differentiation between dead and live cells. Ab1-specific, IgG⁺ single B cells were sorted using a BD FACSARIA™ sorter (BD). Samples were first gated on live (PI⁻), IgG⁺ (FITC⁺) cells, and then displayed on a plot showing the RPE-labeled Ab1 Fab signal on the X-axis and the APC-labeled Ab1^{ctrl} Fab signal on the Y-axis (FIG. 1B).

Single B cells from the RPE⁺, APC⁻ population were literally sorted into a 96-well plate with conditioned medium (rbTSN) and feeder cells and were cultivated (in vitro clonal expansion) for 7 days at 37° C., as previously described (Seeber et al., PLoS One, 9: e86184, 2014) (FIG. 1A). The feeder cells provided CD40 ligand engagement and the rbTSN, made from mitogen-PMA stimulated co-culture of rabbit thymocytes and monocytes, supplied necessary cytokines for B cell proliferation and differentiation (Seeber et al., PLoS One, 9: e86184, 2014).

Optimal B cell culture conditions were observed to be one of the key factors driving individual B cell survival rate and differentiation into antibody-secreting plasma cells. The overall survival rate of IgG⁺ clones was 50-80% across projects, with an average IgG supernatant concentration about 2-3 μg/mL. The supernatants derived from the cultured rabbit B cells were the most essential resources for the following step of primary ELISA screening to confirm clones with desired Ab1 Fab⁺ and Ab1^{ctrl} Fab⁻ phenotype before molecular cloning (FIG. 1A).

### Example 2. Ab1 and Ab1 Framework Control (Ab1^{ctrl}) Fab Preparation and Labeling

Each Ab1^{ctrl} Fab (also referred to as human consensus framework controls) was derived from individual sets of human framework germline genes by selecting the most prevalent amino acid residue at a given position. The CDRs of all human consensus framework controls were mocked with an irrelevant anti-gD tag antibody to guide anti-IDs selection specifically toward Ab1 CDRs. Ab1 framework control Fabs (Ab1^{ctrl} Fab) were designed by grafting the light chain (LC) and heavy chain (HC) complementarity-determining regions (CDRs) of an irrelevant anti-gD mAb (5B6; Genentech) onto four human LC (hIGKV1/V2/V3/V4 or K1/K2/K3/K4) and four human HC (hIGHV1/V2/V3/V4 or H1/H2/H3/H4) consensus frameworks, respectively. Consensus frameworks were determined by selecting the most prevalent amino acid residue at a given position of the human framework germline genes most frequently used in the natural human antibody repertoires (Ippolito et al., PLoS One, 7: e35497, 2012; Lefranc et al., Nucleic Acids Res, 27: 209-212, 1999). A total combination of sixteen Ab1^{ctrl} Fab fragments (KnHn, n=1/2/3/4) were transiently expressed in EXPI293F™ cells (A14528, Thermo Fisher Scientific) and were purified by Protein G affinity chromatography (17088601, GE Healthcare) as reported previously (Bos et al., Biotechnol Bioeng, 112: 1832-1842, 2015).

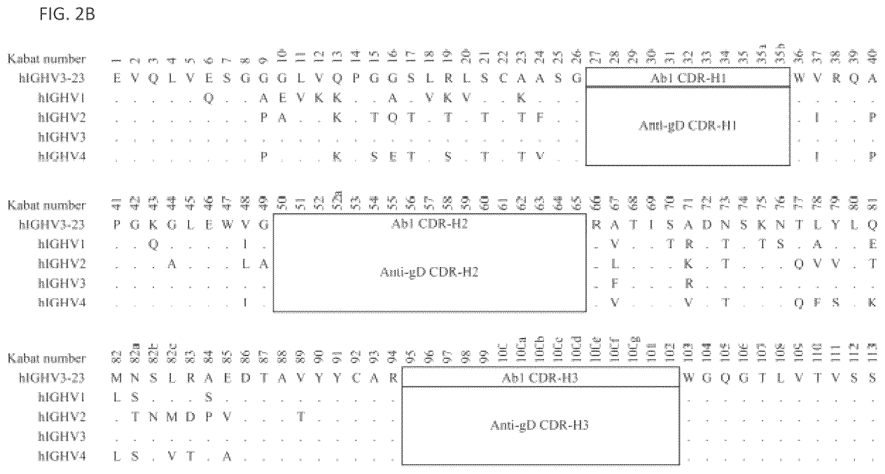

Table 2 shows the Ab1 light chain (LC) and heavy chain (HC) human framework germline from each project. The closest human consensus framework control with the highest sequence identity was chosen to guide the selection. For example, in Project E, the LC (hIGKV1-16) and the HC (hIGHV3-23) of the Ab1 framework germline were aligned with all four designed human LC consensus frameworks (hIGKV1-hIGKV4) and four human HC consensus frameworks (hIGHV1-hIGHV4), respectively, to compare the difference in sequences (FIGS. 2A and 2B). Sequences of consensus framework controls are provided in Table 1. The hIGKV1(K1) and hIGHV3(H3) consensus frameworks had the greatest sequence identity to the Ab1 framework germline LC and HC sequences (100% and 98% identity respectively): thus, a K1H3 consensus framework was chosen as a proper Ab1^{ctrl} Fab for Project E for use as a negative gate to eliminate framework-specific B cells in FACS sorting and a negative control in cultured supernatant primary ELISA screening (FIG. 1A).

Antigen binding fragments of therapeutic IgG antibodies (Ab1 Fab) were prepared by lysyl endopeptidase (129-02541, Wako Chemicals, Inc.) digestion, followed by protein L-agarose column purification (Wranik et al., J Biol Chem, 287: 43331-43339, 2012).

For fluorescence labeling, Ab1 and Ab1^{ctrl} Fab fragments were conjugated with R-phycoerythrin (RPE; 703-0003, Innova Biosciences) and allophycocyanin (APC; 705-0030, Innova Biosciences), respectively, according to the manufacturer's instructions.

The concept of deploying a human consensus framework design, combining 4 human IgG kappa LC families (Kappa 1-Kappa 4) and 4 human HC families (VH1-VH4), was shown here to be highly beneficial, and readily applicable to other antibody families, such as lambda LC and other HC.

### Example 3. High-Throughput Screening to Identify Ab1-Specific Clones for Recombinant Cloning

A robotic system that integrated multi-functional assays in one protocol was established in house to screen Ab1-specific clones in a high-throughput (HTP) manner. The system enabled multiple antigen binding assays to be run simultaneously to handle screening of large panels of B cell culture supernatants (>50 96-well plates) in one day. The advantages of building this system are not only to provide a fast and robust antibody screening platform, but also to eliminate unwanted clones to save downstream processing time.

In some projects (Projects B, D, E, F, G, and I), lower anti-ID yields after primary ELISA screening were observed, possibly due to low immunogenicity of the CDRs for those targets. However, with the power of rabbit single B cell sorting-culture and cloning technology, the weak immune response in these projects was compensated for by delivering a sufficient number of anti-IDs with diversified functions, adequately supplying downstream assay development efforts, as exemplified in the Project E case study. The strong monovalent binding affinities of the majority of anti-IDs generated using this platform, in all 11 projects, ranging from low pM to low nM, were sufficiently sensitive to meet assay performance requirements without further affinity maturation.

A. Ab1 CDR-Specific IgG⁺ B Cell Single-Cell Screening

Following the culturing step of Example 1C, B-cell culture supernatants were transferred via a high-throughput robotic system (BioCel System, Agilent) to a 384-well microplate to screen for Ab1 Fab and Ab1^{ctrl} Fab binding using a standard ELISA protocol as described in Example 1A. Clones having supernatants that bound to the Ab1 Fab (Ab1 Fab⁺) and did not bind to the Ab1^{ctrl} Fab (Ab1^{ctrl} Fab−) were considered to be Ab1 CDRs-specific anti-idiotype clones (Ab2) and were cherry-picked from the original RLT lysis buffer (79216, Qiagen) treated source plates for molecular cloning.

By incorporating a negative-selection step with the human consensus framework designed to mimic the therapeutic drug's framework, the ability to selectively produce highly specific anti-IDs against the unique amino acid sequence of therapeutic-drug CDRs is considerably enhanced. Anti-IDs specifically directed to the CDRs of monoclonal antibody therapeutic drugs (often humanized or human antibodies) are less prone to interference from excess amounts of endogenous human immunoglobulins having similar Ig framework to the Ab1 present in biological matrices such as serum.

In Project E, thousands of clones' cultured supernatants were subjected to primary ELISA screening, and the majority of clones were positive against the Ab1 Fab (OD>0.25), but not the Ab1^{ctrl} Fab (OD<0.1) (FIG. 1D). The top 34 Ab2 clones, which demonstrated a significantly strong signal in the assay for Ab1 Fab binding (OD>1) and a relative 10-fold lower signal in the assay for Ab1^{ctrl} Fab binding (OD<0.1), were selected for molecular cloning, as described below.

B. Ab2 Molecular Cloning, Sequence Analysis, and Expression

For molecular cloning of selected Ab2, first, total RNAs of Ab2 clones were isolated using the NUCLEOSPIN® 96 RNA Core Kit (740466.4, Macherey-Nagel) according to the manufacturer's instructions. cDNA was prepared by reverse transcription of the mRNA from total RNA using SUPERSCRIPT™ III First-Strand Synthesis SuperMix (18080400, INVITROGEN™). The V regions of individual rabbit B-cells were amplified through PCR reaction using ACCUPRIME™ Pfx SuperMix (12344040, INVITROGEN™) with forward and reverse primers designed to target V_{L} and V_{H} regions, as previously described (Seeber et al., PLoS One, 9: e86184, 2014). The PCR products of VL and VH were then cleaned up using the NUCLEOSPIN® 96 Extract II kit (740658.1, Macherey-Nagel) and cloned into expression vectors encoding the rabbit IgG LC and HC constant regions, respectively, using the IN-FUSION® HD ECODRY™ Cloning Kit (638915, Takara). The plasmid DNAs were then purified using NUCLEOSPIN® 96 Plasmid Mini Kit (740616.4, Macherey-Nagel) for sequence analysis (determined by the dynamic programming alignment algorithm at each framework and CDR regions) and for transfection to express recombinant rabbit IgGs (Bos et al., Biotechnol Bioeng, 112: 1832-1842, 2015).

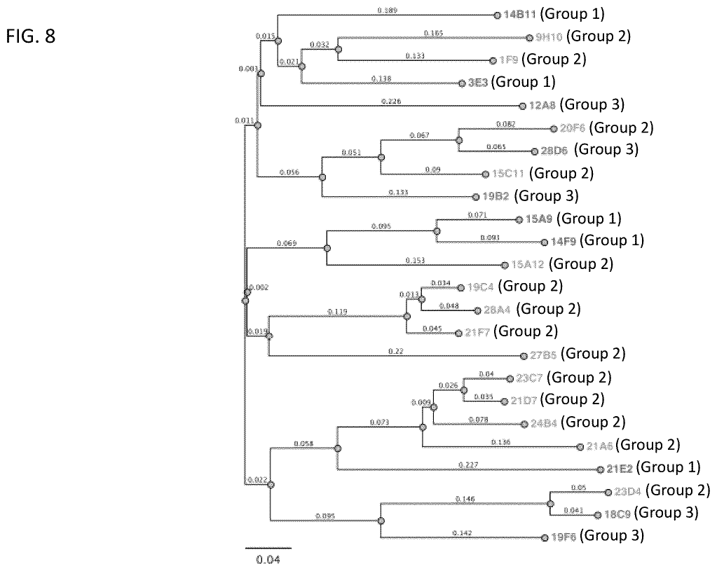

For the sequence diversity analysis in Project E, the CDR regions of both the VH and the VL of the 24 unique clones were individually extracted and subsequently concatenated into a sequential residue string for each unique clone. The 24-residue strings were then aligned using Clustal W with both gap opening and extension penalties set at zero equivalent to local alignment (Larkin et al, Bioinformatics, 23: 2947-2948, 2007) (FIGS. 2A and 2B). In order to visualize the sequence similarity of the 24 clones, the sequence alignment result was referenced to use with Neighbor Joining method to generate an unrooted phylogenetic tree, as shown in FIG. 8. It can be observed that two clades towards the bottom of the tree consist predominantly of group 2 clones, suggesting that these could be affinity matured variants originating from common ancestral clones. On the other hand, both group 1 and group 3 clones tend to be derived independently, resulting in scattered locations along the tree.

C. ELISA Assay for Ab2 Binding Affinity

The recombinant IgGs of Ab2 clones expressed after cloning were reconfirmed for Ab1 specificity by ELISA. To measure the binding affinity of Ab2 rabbit antibody (rAb) clones, surface plasmon resonance (SPR) assays were performed using a Biacore™-T200 instrument (GE Healthcare). A Series S sensor chip Protein A (29127555, GE Healthcare) was applied to capture each Ab2 clone on a different flow cell (FC) to achieve approximately 100 response units (RU), followed by the injection of five-fold serial dilutions of Ab1 Fab (0.03 nM to 100 nM) in HBS-EP buffer (100 mM HEPES pH 7.4, 150 mM NaCl, 3 mM EDTA, 0.05% (v/v) Surfactant P20) with a flow rate of 50 μl/minute at 25° C. Association rates (kₒₙ) and dissociation rates (k_{off}) were calculated using a simple one-to-one Langmuir binding model (Biacore T200 evaluation software version 2.0). The equilibrium dissociation constant (K_{D}) was calculated as the ratio k_{off}/kₒₙ.

In Project E, the recombinant IgGs of 24 unique Ab2 clones expressed after cloning were assessed for Ab1 specificity as described above and showed positive binding against Ab1 Fab (OD>0.3) and nearly undetectable binding against any control Fab, including the designed Ab1^{ctrl} Fab (K1H3) and other native IgG Fab fragments derived from normal human plasma IgGs (Hu^{ctrl} Fab; 401116, Sigma) (OD<0.05) (FIGS. 1A and 1E).

### Example 4. Anti-IDs Affinity and Epitope Characterization

A. Anti-ID Affinity

High affinity anti-IDs are a desirable tool in assay development for antibody drugs (Ab1). For example, in a bridging assay, a low coating density of anti-IDs as capture reagents is required to avoid potential Ab1 binding to the surface with both arms, which would reduce the assay sensitivity; the affinity of the anti-IDs is a determinant to meet this requirement.

To measure the binding affinity of Ab2 rAb clones, surface plasmon resonance (SPR) assays were performed using a Biacore™-T200 instrument (GE Healthcare). A Series S sensor chip Protein A (29127555, GE Healthcare) was applied to capture each Ab2 clone on a different flow cell (FC) to achieve approximately 100 response units (RU), followed by the injection of five-fold serial dilutions of Ab1 Fab (0.03 nM to 100 nM) in HBS-EP buffer (100 mM HEPES pH 7.4, 150 mM NaCl, 3 mM EDTA, 0.05% (v/v) Surfactant P20) with a flow rate of 50 μl/minute at 25° C. Association rates (kₒₙ) and dissociation rates (k_{off}) were calculated using a simple one-to-one Langmuir binding model (Biacore T200 evaluation software version 2.0). The equilibrium dissociation constant (K_{D}) was calculated as the ratio k_{off}/kₒₙ.

Six of the eleven unique Ab1 anti-IDs projects described herein yielded antibodies having single-digit pM affinity, and the remaining projects yielded antibodies having affinity in the mid to high pM affinity range (Table 2). In Project E, the majority of anti-IDs (22 out of 24) had affinities against Ab1 Fab of <0.5 nM, with the best clone (14F9) having an affinity of 4 pM (Table 3).

TABLE 3
Anti-IDs binding affinity and epitope type
characterization summary in project E

| | | | Ab2 epitope type | | |
|---|---|---|---|---|---|
| | | Affinity to Ab1 | Ag | Ag non- | Ag + Ab1 |
| Anti-IDs (Ab2) | | [K_{D}: nM] | blocking | blocking | complex |
| Group 1 | 14F9 | 0.004 | No | Yes | Yes |
| | 14B11 | 0.028 | No | Yes | Yes |
| | 3E3 | 0.079 | No | Yes | Yes |
| | 21E2 | 0.083 | No | Yes | Yes |
| | 15A9 | 0.126 | No | Yes | Yes |
| Group 2 | 2ID7 | 0.021 | No | Yes | No |
| | 1F9 | 0.023 | No | Yes | No |
| | 21A6 | 0.031 | No | Yes | No |
| | 23C7 | 0.055 | No | Yes | No |
| | 24B4 | 0.072 | No | Yes | No |
| | 9H10 | 0.073 | No | Yes | No |
| | 19C4 | 0.088 | No | Yes | No |
| | 23D4 | 0.098 | No | Yes | No |
| | 15A12 | 0.101 | No | Yes | No |
| | 28A4 | 0.108 | No | Yes | No |
| | 21F7 | 0.129 | No | Yes | No |
| | 20F6 | 0.131 | No | Yes | No |
| | 15C11 | 0.252 | No | Yes | No |
| | 27B5 | 0.366 | No | Yes | No |
| Group 3 | 18C9 | 0.167 | Yes | No | No |
| | 28D6 | 0.432 | Yes | No | No |
| | 12A8 | 0.351 | Yes | No | No |
| | 19B2 | 5.688 | Yes | No | No |
| | 19F6 | 4.517 | Yes | No | No |

Twenty-four unique anti-IDs identified in project E are listed and ranked by their Biacore SPR affinity against Ab1 Fab (best to worst). The epitope type of each clone is included. In total, there are 3 groups of anti-IDs; Group 1: Ag non-blocking with Ag + Ab1 complex specificity (5 clones), Group 2: Ag non-blocking without Ag + Ab1 complex specificity (14 clones), Group 3: Ag blocking (5 clones).

B. Epitope Characterization

As Ab1 may interact with soluble or shed target molecules (antigens; hereafter referred to as Ag) in serum, there may be several forms of Ab1 present in circulation: either free, partially bound, or fully bound. Therefore, it is important to proactively establish what forms of Ab1 are to be measured in a given assay, particularly when the molar concentration of Ag compared to Ab1 is not negligible at the timepoints of therapeutic concentration measurements, such as in Project E.

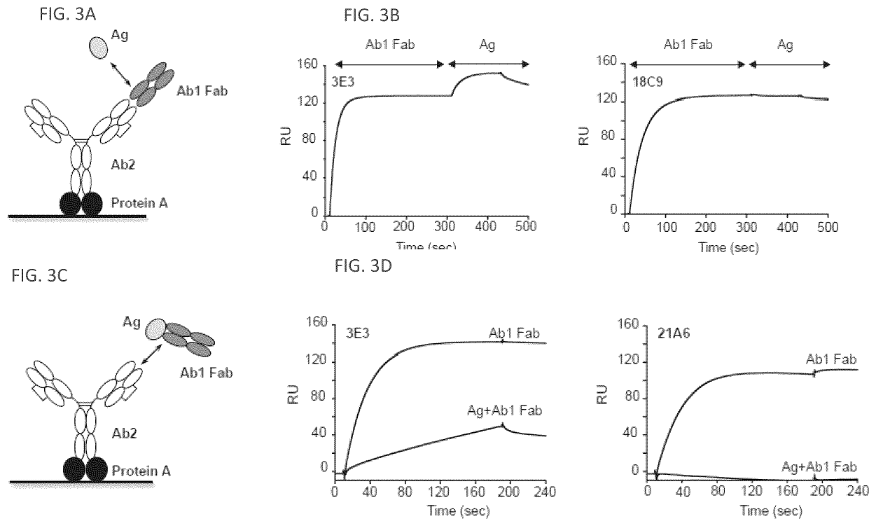

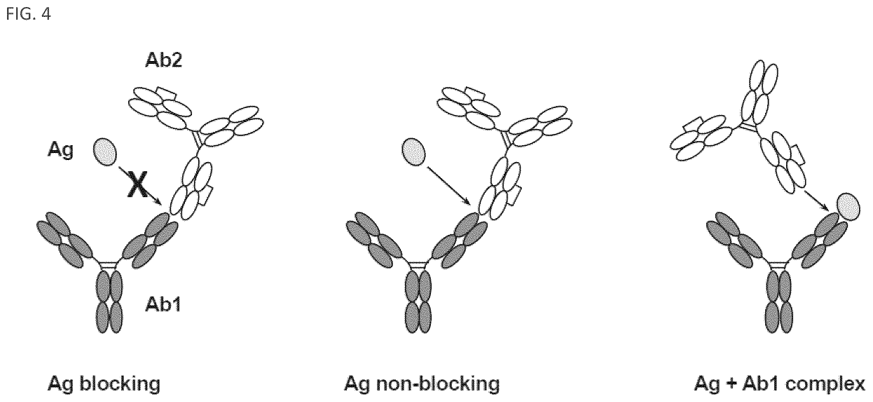

Anti-ID epitope types were classified according to their activity in the presence of Ag as Ag blocking, Ag non-blocking, and Ag+Ab1 complex using high sensitivity and high-throughput Biacore™ SPR (FIG. 4). The Ag blocking type are anti-IDs that bind to the paratope of Ab1 and interfere with Ag binding, thereby allowing detection of free Ab1 only (e.g., 18C9 in FIG. 3B). In contrast, an Ag non-blocking type anti-ID binds outside the paratope of Ab1 and still allows Ag to bind Ab1, and therefore can be used to detect free, and possibly partially bound and fully bound Ab1 (e.g., 3E3 in FIG. 3B).

To determine the epitope type of each Ab2 clone, the format as described above (FIG. 4) was used to capture an individual Ab2 rabbit IgG on each FC. For the antigen (Ag) blocking or non-blocking epitope study, 100 nM Ab1 Fab was first injected for 5 minutes to reach saturation, followed by a second injection of 50 nM Ag for 3 minutes to detect binding (FIG. 3A). The interactions between Ab1 Fab to Ab2 and the subsequent Ag to Ab1 Fab were recorded separately to calculate the differences in binding response (Ag-Ab1 Fab), as well as the theoretical Ag binding Rmax. Both factors were then used to determine the actual Ag binding Rmax in percentage, which is >0 for an Ag non-blocking epitope and for an Ag-blocking epitope (Table 4). For the Ag and Ab1 complex-specific epitope study, the binding Rmax of 500 nM Ag+50 nM Ab1 Fab complex (premixed with 10-fold excess of Ag to saturate Ab1 Fab) and 50 nM Ab1 Fab only against a similar level of Ab2 captured by protein A were recorded (FIG. 3C), and the ratio between them was calculated as a percentage. If the value was >10%, the Ab2 was considered to be of the Ag+Ab1 complex epitope type; otherwise, it belonged to a non Ag+Ab1 complex epitope type (Table 5).

To confirm whether the Ag non-blocking type of anti-IDs can also recognize the bound form of Ab1, Ag+Ab1 complexes (with 10-fold excess of Ag to saturate Ab1 binding sites) were generated to determine the binding compared to Ab1 only. 5 out of 19 anti-IDs were capable of binding Ag+Ab1 complexes (e.g., 3E3 in FIG. 3D).

TABLE 4
Anti-IDs Ag blocking or non-blocking epitope type determination in project E

| | | | Ag | Ag -Ab1 | Theoretical | | Ab2 |
|---|---|---|---|---|---|---|---|
| | Affinity to | | binding | binding | Ag binding | Actual Ag | epitope type |
| Anti-IDs | Ab1 Fab | Ab1 Fab binding | response | response | Rmax | binding | (Ag |
| (Ab2) | [K_{D}: nM] | response [RU] | [RU] | [RU] | [RU] | Rmax [%] | blocking) |
| 3E3 | 0.079 | 128 | 152 | 24 | 64 | 38 | No |
| 21E2 | 0.083 | 115 | 126 | 11 | 58 | 19 | No |
| 15A12 | 0.101 | 125 | 136 | 11 | 63 | 18 | No |
| 14B11 | 0.028 | 138 | 150 | 12 | 69 | 17 | No |
| 14F9 | 0.004 | 128 | 137 | 9 | 64 | 14 | No |
| 9H10 | 0.073 | 95 | 101 | 6 | 48 | 13 | No |
| 19C4 | 0.088 | 124 | 132 | 8 | 62 | 13 | No |
| 23D4 | 0.098 | 128 | 136 | 8 | 64 | 13 | No |
| 21F7 | 0.129 | 120 | 128 | 8 | 60 | 13 | No |
| 20F6 | 0.131 | 124 | 132 | 8 | 62 | 13 | No |
| 27B5 | 0.366 | 132 | 140 | 8 | 66 | 12 | No |
| 1F9 | 0.023 | 122 | 129 | 7 | 61 | 11 | No |
| 21A6 | 0.031 | 140 | 148 | 8 | 70 | 11 | No |
| 24B4 | 0.072 | 131 | 138 | 7 | 66 | 11 | No |
| 28A4 | 0.108 | 128 | 135 | 7 | 64 | 11 | No |
| 15C11 | 0.252 | 110 | 116 | 6 | 55 | 11 | No |
| 23C7 | 0.055 | 149 | 154 | 5 | 75 | 7 | No |
| 21D7 | 0.021 | 184 | 189 | 5 | 92 | 5 | No |
| 15A9 | 0.126 | 155 | 157 | 2 | 78 | 3 | No |
| 18C9 | 0.167 | 126 | 126 | 0 | 63 | 0 | Yes |
| 28D6 | 0.432 | 110 | 110 | 0 | 55 | 0 | Yes |
| 12A8 | 0.351 | 146 | 144 | −2 | 73 | −3 | Yes |
| 19B2 | 5.688 | 117 | 111 | −6 | 59 | −10 | Yes |
| 19F6 | 4.517 | 121 | 101 | −20 | 61 | −33 | Yes |

The binding response of Ab1 Fab against each protein A-captured Ab2 clone, following Ag binding to Ab1 Fab was recorded. The actual Ag binding response (Ag - Ab1 Fab) was used to determine the actual Ag binding Rmax [%] by dividing it with the theoretical Ag binding Rmax;
$Rmax = \frac{molecular weight of Ag}{molecular weight of Ab 1 Fab} \times ( Ab 1 Fab binding response ) \times ( Ab 1 Fab stoichiometry ) ;$ The Ag blocking or non-blocking epitope type of Ab2 is defined by the actual Ag binding Rmax [%], which is >0 for Ag non-blocking and ≤0 for Ag blocking.
All twenty-four unique anti-IDs identified in project E are listed and ranked by their actual Ag binding Rmax [%] (highest to lowest).

TABLE 5
Anti-IDs Ag and Ab1 complex epitope type
determination in project E

| | Ag + | | (Ag + Ab1 | |
|---|---|---|---|---|
| Anti- | Ab1 Fab | Ab1 Fab | Fab)/Ab1 | |
| IDs | binding | binding | Fab binding | Ab2 epitope type |
| (Ab2) | Rmax [RU] | Rmax [RU] | Rmax [%] | (Ag + Ab1 complex) |
| 3E3 | 47 | 143 | 33 | Yes |
| 14B11 | 25 | 147 | 17 | Yes |
| 21E2 | 26 | 194 | 13 | Yes |
| 14F9 | 17 | 145 | 12 | Yes |
| 15A9 | 17 | 164 | 10 | Yes |
| 20F6 | 7 | 124 | 6 | No |
| 15A12 | 6 | 123 | 5 | No |
| 23D4 | 6 | 138 | 4 | No |
| 27B5 | 6 | 139 | 4 | No |
| 21D7 | 6 | 162 | 4 | No |
| 19B2 | 3 | 106 | 3 | No |
| 1F9 | 4 | 142 | 3 | No |
| 12A8 | 2 | 164 | 1 | No |
| 23C7 | 2 | 172 | 1 | No |
| 21A6 | 0 | 155 | 0 | No |
| 24B4 | −1 | 151 | −1 | No |
| 15C11 | −5 | 120 | −4 | No |
| 19C4 | −7 | 136 | −5 | No |
| 28D6 | −7 | 129 | −5 | No |
| 18C9 | −6 | 115 | −5 | No |
| 28A4 | −10 | 157 | −6 | No |
| 21F7 | −9 | 138 | −7 | No |
| 9H10 | −7 | 94 | −7 | No |
| 19F6 | −9 | 119 | −8 | No |

Under similar protein A capturing level for each Ab2 clone, the binding Rmax of Ag + Ab1 Fab as well as Ab1 Fab were recorded. The binding Rmax ratio between Ag + Ab1 Fab and Ab1 Fab was then calculated as %. If the value is >10%, the clone is considered Ag and Ab1 complex specific epitope type. All twenty-four unique anti-IDs identified in project E are listed and ranked by their binding Rmax ratio
$( \frac{( Ag + Ab 1 Fab )}{Ab 1 Fab} ) [ \% ] ( highest to lowest ) .$

C. Ab2 Rabbit Antibody Epitope Binning Using the Carterra SPR

Overall, all 24 of the unique anti-IDs in Project E can be categorized into three groups: Group 1: Ag non-blocking with specificity to Ag+Ab1 complex (5 clones); Group 2: Ag non-blocking without specificity to Ag+Ab1 complex (14 clones); Group 3: (5 clones). Results indicate that group 1 anti-IDs can be functionally suitable for total Ab1 detection, whereas group 2 and 3 anti-IDs can be used only for free Ab1 detection, and differ in whether they interfere with Ag binding.

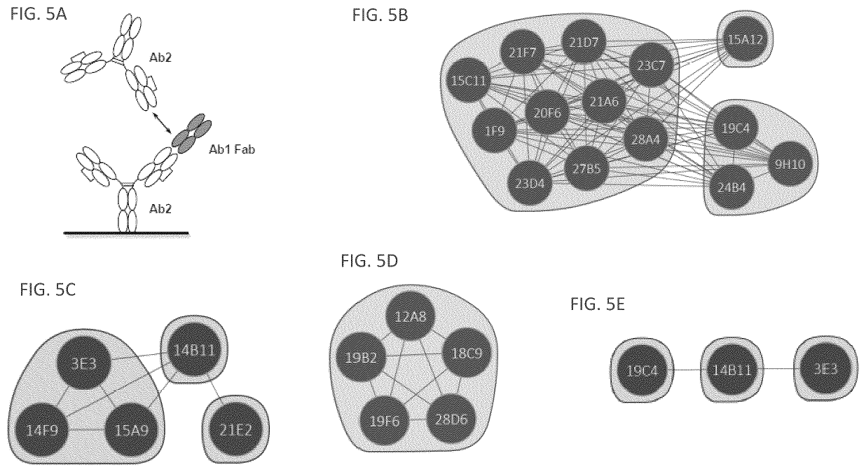

To elucidate the subtle epitope differences in each group of anti-IDs, pairwise competition experiments were performed by high-throughput Carterra SPR microfluidics under the classic sandwich format (FIG. 5A). A microarray-based 96×96 microfluidic system (IBIS-MX96 SPRi, Carterra USA) was used for the Ab2 rAb epitope binning experiment. First, each Ab2 rabbit IgG (10 μg/mL in 10 mM sodium acetate buffer pH 4.5) was directly immobilized onto a sensorprism CMD 200M sensor chip (CMD 200M, XanTec Bioanalytics, Germany) using amine-coupling chemistry in a continuous flow microspotter (CFM, Carterra, USA). Next, 100 nM Ab1 Fab was injected over the sensor chip for 4 minutes binding, followed by another 4-minute injection of each Ab2 rabbit IgG (10 μg/mL in HBS-EP buffer) at 25° C. The chip surface was regenerated between each cycle using 10 mM Glycine pH 1.5, and the binding response was recorded and analyzed in Carterra microfluidics' binning software for heat map generation and network plotting. The competing relationships (cords) between Ab2 (nodes) allow the anti-IDs to be clustered into bins (inscribed by the envelopes), where a bin represents a family of anti-IDs sharing an identical blocking profile when tested against the other anti-IDs (FIGS. 5B-5D).

In the group 1 anti-IDs, there were three bins evidenced by this study, where clone 14611 binds to a bridging epitope between clones 21E2, 3E3, and the others (FIG. 5C). In the group 2 anti-IDs, a similar result was observed, except for a much larger group of anti-IDs (10 out of 14) targeting a bridging epitope (FIG. 5B). In group 3 anti-IDs, interestingly, only one bin was identified (FIG. 5D).

### Example 5. PK and ADA Assay Development Using Anti-IDs

For the development of pharmacokinetics (PK) and anti-drug antibody (ADA) assays in Project E, five anti-IDs were selectively chosen from group 1 (3E3 and 14611; aiming to detect total Ab1) and group 2 (9H10, 19C4 and 2464; aiming to detect free Ab1 in the presence of Ag interference) to manage the scale of functional assay development, as Ag was present at a non-negligible concentration compared to Ab1 in serum samples at the time of measurement.

Having a panel of well-characterized anti-IDs can be advantageous for the development of both clinical PK and ADA assays in a number of ways. For PK assays, availability of multiple anti-ID clones allows for assessment and comparison between various assay formats, including the anti-ID/anti-ID bridging format described herein. This format, which is preferred for development of free-drug PK assays, can improve both the sensitivity and the specificity of the assay over the use of generic, non-drug specific reagents (Kelley et al., AAPS J., 9(2): E156-E163, 2007). Furthermore, the use of specific reagents ensures a robust dose-response curve covering a wide dynamic range of the assay, while maintaining acceptable accuracy and precision (DeSilva et al., Pharm Res, 20(11): 1885-1900, 2003). Often, clones sharing similar characteristics may perform differently in the assay (as demonstrated with clones 9H10 and 2464). This can be due to varying degrees of plate-coating efficiency between anti-IDs, structural changes introduced during the conjugate-formation process, or matrix effects (e.g., interfering factors present in blood, plasma serum, etc.). Therefore, an adequately diverse panel of clones available for screening and selection is a critical element of successful anti-ID reagent production programs. Epitope characterization and grouping can further inform assay development decisions and provide a choice between development of a total-drug assay or a free-drug assay, thus affecting study data interpretation.

Selection criteria of the anti-IDs for an ADA assay, on the other hand, are not as rigorous as those for PK assays. Beyond controlling an assay's performance over time, as a surrogate positive ADA source, the anti-ID is used to demonstrate and assess important assay parameters during validation, such as sensitivity, specificity, drug tolerance, precision, and analyte stability. Additionally, anti-IDs may also be used to characterize antibody responses against particular epitopes on the Ab1 through use in competition assays.

A. Ab2 Development for PK Assays

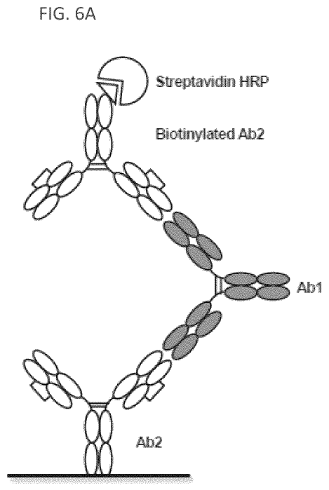

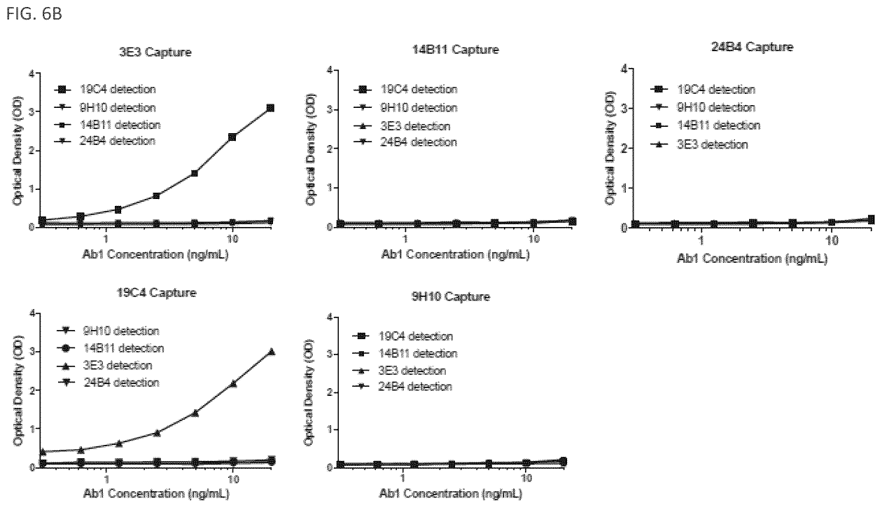

For PK assay development, a sandwich ELISA format was used (FIG. 6A). First, 96-well microtiter plate was separately coated with each Ab2 rAb clone (1 μg/mL) overnight at 4° C. Two-fold serial dilutions of the therapeutic antibody Ab1 (20 ng/mL to 0.3 ng/mL) in assay buffer (1× PBS, 0.5% BSA, and 0.05% polysorbate 20) containing 2% pooled normal human serum were then added to the plate and incubated for 2 hours. To detect bound Ab1, a biotinylated version of each Ab2 clone (0.2 μg/mL; 10:1 biotinylation ratio) was added, followed by streptavidin HRP conjugate (DY998, R&D Systems) and TMB substrate (5120-0047, KPL, Inc.) for color development. The plate reaction was stopped by adding 1 M phosphoric acid, and absorbance was read at 450 nm with a 630 nm reference wavelength.

Using the sandwich ELISA format shown in FIG. 6A, 3E3 (group 1) and 19C4 (group 2) were identified as forming a suitable antibody pair to capture and detect Ab1 (FIG. 6B). The pair displayed a high signal-to-noise ratio and robust dose-response curve titration in either orientation. As evidenced in the epitope binning characterization studies, 3E3 and 19C4 have distinct and non-overlapping epitopes, allowing for both reagents to sandwich Ab1 (FIG. 5E). In contrast, clones 14611 (same group 1 as 3E3), 9H10 and 2464 (same group 2 as 19C4) did not demonstrate compatibility with either 3E3 or 19C4. Clones 9H10 and 2464 have non-overlapping epitopes with clone 3E3; however, neither offer the same robust dose-response curve when paired with 3E3 as 19C4 does, despite sharing similar characteristics with 19C4. These results underscore the importance of experimental bioanalytical data in this type of reagent selection process, complementing the reagent characterization data. While coating the plate with either 3E3 or 19C4 and using the remaining clone as a detection reagent results in an acceptable assay, 3E3 was chosen for use as the capture antibody due to its better signal-to-noise ratio. More importantly, this reagent-pair also afforded better detection and quantitation of free and bound drug (drug complexed with the circulating target antigen). This would be considered a total drug assay, whereas an assay with the opposite orientation would be considered a free drug assay and be subject to increased interference by the presence of circulating targets.

B. Ab2 Development for ADA Assays

A sensitive assay to detect the presence of anti-drug antibodies (ADAs) in treated patients is critically important for evaluating immune responses to recombinant therapeutics. For ADA assay development, anti-IDs should preferably target epitopes that are unique for drug molecules. Antibodies against Ab1 derived from B-cells using the production and selection strategy described herein are most likely anti-IDs. Therefore, the aforementioned five clones were investigated for suitability as a surrogate positive control in a clinical immunogenicity assay for Project E.

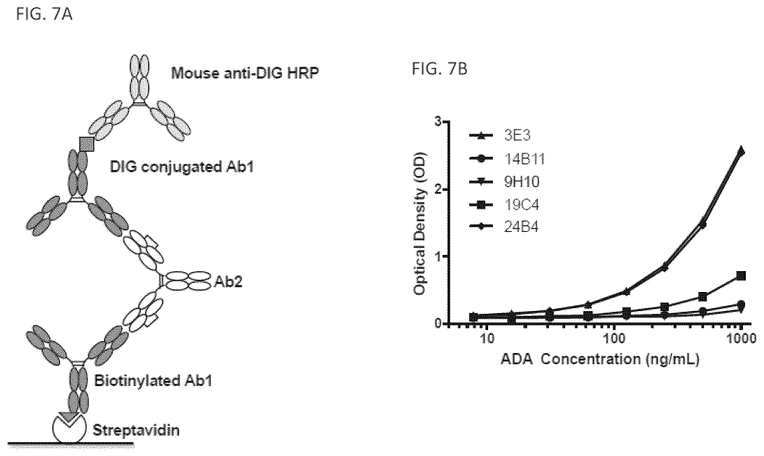

For ADA assay development, a bridging ELISA format was used (FIG. 7A). The ADA samples were prepared by adding 1000 ng/mL of an individual Ab2 clone to neat human serum from healthy donors. The samples were diluted 1/20, followed by two-fold serial dilution to produce a titration curve (1000 ng/mL to 7.8 ng/mL). Biotinylated and digoxigenin-conjugated therapeutic Ab1 antibody reagents were added (each at 4 μg/mL; 10:1 challenge ratios) and incubated overnight with the diluted sample, forming an immune complex with Ab2. Thereafter, the complex was captured onto a streptavidin-coated microtiter plate (11734776001, Roche), followed by detection using a mouse anti-DIG HRP conjugated mAb (200032156, Jackson Immunoresearch) and color development as described above for the PK assay.

Both 3E3 and 24B4 produced a specific and robust binding curve, with superior characteristics over the other anti-IDs, and are thus suitable anti-ID reagents for ADA assay development in this project (FIG. 7B).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method of identifying one or more antibodies that bind to a desired region of a target protein, the method comprising:
(a) providing a sample from an animal that has been immunized with the target protein or a fragment thereof comprising the desired region, wherein the sample contains IgG⁺ B cells;
(b) enriching the sample for IgG⁺ B cells by separating the IgG⁺ B cells from one or more undesired cell types in the sample, wherein the separating comprises:
(i) contacting the sample with one or more antibodies or antibody fragments that bind to the one or more undesired cell types, wherein the one or more antibodies or antibody fragments comprise a tag; and
(ii) contacting the sample with a surface having affinity for the tag, wherein the one or more undesired cell types bound to the one or more antibodies or antibody fragments are retained on the surface, thereby separating the IgG⁺ B cells from the one or more undesired cell types and enriching the sample for IgG⁺ B cells;
(c) culturing the separated IgG⁺ B cells of step (b) individually; and
(d) identifying one or more IgG⁺ B cells that produce antibodies that bind to the desired region of the target protein, the identifying comprising assessing the affinity of supernatants of individually cultured IgG⁺ B cells of step (c) for both:
(i) the target protein or a fragment thereof comprising the desired region; and
(ii) a control protein comprising one or more undesired binding sites of the target protein or a non-target protein;
wherein supernatants that have affinity for the target protein or fragment thereof and do not have affinity for the control protein identify IgG⁺ B cells producing antibodies that bind to the desired region of the target protein.

2. The method of claim 1, wherein the animal is a rabbit or a rat.

3. The method of claim 1, wherein the sample is a blood sample, a serum sample, or a peripheral blood mononuclear cell (PBMC) sample.

4. The method of claim 1, wherein the animal has been immunized for about 8 weeks.

5. The method of claim 1, wherein the sample has been processed to remove macrophages and monocytes.

6. The method of claim 1, wherein the undesired cell types are one or more of IgM B cells, myeloid cells, and T cells.

7. The method of claim 6, wherein:
(a) the one or more antibodies or antibody fragments that bind to IgM B cells are one or more anti-IgM antibodies or antibody fragments thereof that bind IgM;
(b) the one or more antibodies or antibody fragments that bind to myeloid cells are one or more anti-CD11 b antibodies or antibody fragments thereof that bind CD11 b; and/or
(c) the one or more antibodies or antibody fragments that bind to T cells are anti-T-lymphocyte antibodies or antibody fragments thereof that bind T-lymphocytes.

8. The method of claim 1, wherein the one or more antibodies or antibody fragments that bind to the one or more undesired cell types comprise a biotin tag and the surface comprises streptavidin.

9. The method of claim 1, wherein the surface is a bead.

10. The method of claim 9, wherein the bead is a magnetic bead.

11. The method of claim 1, wherein step (b) further comprises (iii) contacting the enriched sample with an antibody or antibody fragment that comprises a first marker and binds to IgG⁺ B cells and an agent that identifies viable cells.

12. The method of claim 11, wherein the antibody or antibody fragment that binds to IgG⁺ B cells is an anti-IgG antibody and/or the agent that identifies viable cells is propidium iodide.

13. The method of claim 11, wherein step (b)(iii) further comprises contacting the sample with the target protein or a fragment thereof comprising the desired region, wherein the target protein or fragment thereof comprises a second marker.

14. The method of claim 13, wherein step (b)(iii) further comprises contacting the sample with a control protein comprising one or more undesired binding sites of the target protein, wherein the control protein comprises a third marker.

15. The method of claim 14, wherein the first marker, second marker, and third marker are fluorescent markers.

16. The method of claim 14, wherein step (b) further comprises (iv) isolating cells that are identified as viable by the agent that identifies viable cells and that comprise the first and second markers, but not the third marker.

17. The method of claim 16, wherein the isolating is by multi-parameter fluorescence activated cell sorting (FACS).

18. The method of claim 1, wherein, in step (d), an ELISA is performed for assessing the affinity of supernatants for both (i) the target protein or fragment thereof and (ii) the control protein.

19. The method of claim 1, further comprising (e) cloning the VH and VL regions of one or more IgG⁺ B cells that have been identified as producing antibodies that bind to the desired region of the target protein.

20. The method of claim 1, wherein the target protein is an antibody or an antibody fragment.

21. The method of claim 20, wherein the desired region of the antibody or antibody fragment is a complementarity determining region (CDR).

22. The method of claim 20, wherein the animal has been immunized with a fragment of the antibody comprising the desired region.

23. The method of claim 22, wherein the fragment of the antibody comprising the desired region is an antigen-binding fragment (Fab).

24. The method of claim 20, wherein the one or more undesired binding sites of the target protein are one or more framework regions of the antibody or antibody fragment.

25. The method of claim 20, wherein the control protein comprising one or more undesired binding sites of the target protein is a Fab fragment comprising:
(i) a light chain (LC) comprising a framework region having at least 85% identity to the LC framework region of the target protein and a set of irrelevant LC CDRs; and
(ii) a heavy chain (HC) comprising a framework region having at least 85% identity to the HC framework region of the target protein and a set of irrelevant HC CDRs.

26. The method of claim 25, wherein the irrelevant LC and HC CDRs are the CDRs of an anti-gD monoclonal antibody (mAb).

27. The method of claim 26, wherein the anti-gD mAb is 5B6.

28. The method of claim 1, wherein the target protein is not an antibody or an antibody fragment.

29. The method of claim 28, wherein the desired region of the target protein is a domain of the target protein.

30. The method of claim 28, wherein step (d) comprises assessing the affinity of supernatants of individually cultured IgG⁺ B cells of step (c) fora fragment of the target protein comprising the desired region.

31. The method of claim 30, wherein the fragment of the target protein comprising the desired region is linked to an irrelevant protein.

32. The method of claim 28, wherein the control protein of step (d) is:
(i) a version of the target protein that is devoid of the desired region;
(ii) a protein that is related to the target protein and does not comprise the desired region; or
(iii) an irrelevant control protein.

33. The method of claim 1, wherein a plurality of antibodies that bind to a desired region of a target protein is produced.

34. The method of claim 33, wherein at least 100, 500, 1000, 10000, 20000, or 30000 antibodies are produced.

35. The method of claim 33, wherein at least 50% of the antibodies produced are unique.

36. The method of claim 33, wherein the plurality of antibodies binds the desired region of the target protein with a K_{D} of about 200 nM or lower; about 50 nM or lower; about 10 nM or lower; about 1 nM or lower; about 0.1 nM or lower; or about 0.01 nM or lower.

37. The method of claim 33, wherein the target protein is an antibody or an antibody fragment and the plurality of antibodies comprises at least one antigen-blocking antibody.

38. The method of claim 33, wherein the target protein is an antibody or an antibody fragment and the plurality of antibodies comprises at least one antigen non-blocking antibody.

39. The method of claim 38, wherein the antigen non-blocking antibody binds to an antigen-antibody complex.

40. The method of claim 1, wherein the IgG⁺ B cells of step (c) have increased viability relative to IgG⁺ B cells that have been isolated using a method that does not comprise a step of enriching the sample for IgG⁺ B cells according to claim 1.

41. The method of claim 19, wherein steps (a)-(e) are performed within twelve weeks.
